(19) United States
(12) Patent Application Publication
(10) Pub. No.: US 2009/0092624 A1
(43) Pub. Date: Apr. 9, 2009

(54) Antiinfective Flavononol Compounds and Methods of Use Thereof

(76) Inventors: Randall S. Alberte, Estero, FL (US); William P. Roschek, JR., Naples, FL (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 12/192,759

(22) Filed: Aug. 15, 2008

Related U.S. Application Data
(60) Provisional application No. 60/956,512, filed on Aug. 17, 2007.

(51) Int. Cl. A61K 39/00; C07D 215/02; C07D 215/38; C07D 335/04; C07D 311/04; A41D 13/00; A62B 7/10; B01D 53/22; A61P 31/04; A61K 31/47; A61K 31/382; A61K 31/352

(52) U.S. Cl. 424/184.1; 546/153; 546/159; 549/23; 549/399; 549/404; 514/312; 514/313; 514/432; 514/456; 435/7.1; 2/243.1; 2/69; 128/846; 96/14; 210/500.28; 128/205.27

(57) ABSTRACT

The present invention relates in part to antiinfective flavononol compounds represented by formula I:

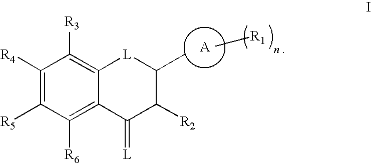

Another aspect of the invention is a method for treating an infection in a subject by administering the compounds of Formula I to the subject.

### RELATED APPLICATIONS

This application claims the benefit of priority to U.S. Provisional Application Ser. No. 60/956,512, filed on Aug. 17, 2007, which is herein incorporated by reference in its entirety.

### BACKGROUND OF THE INVENTION

Infections caused by or related to microbial agents are a major cause of human illness worldwide, and the frequency of resistance to standard antiinfective agents has risen dramatically over the last decade. Infective agents include but are not limited to bacteria, viruses, fungi, protozoans, and prions.

For example, methicillin resistant Staphylococcus aureus (MRSA) have become a major public health concern. Increasing numbers of individuals, and particularly the young and elderly, test positive for MRSA strains of this Gram positive bacterium common to blood stream infections, cutaneous infections and medical device biofilms. Antibiotic resistance is also common in Gram negative bacteria including entercocci and Pseudomonas aeruginosa. The entercocci are causative agents of many gastrointestinal tract disorders, and stains of vancomycin-resistant Enterococcus faecalis and E. faecium (VRE) have become common in processed foods and meat, and in public bathing areas (Yesim Cetinkaya, Pamela Falk, and C. Glen Mayhall, 2000. Clin. Microbiol. Rev. 13:686-707). Pseudomonas aeruginosa infections of the upper respiratory tract is the major cause of morbidity and mortality in adult patients with cystic fibrosis (CF) (Hoiby, N., and C. Koch. Thorax, 1990, 45:881-884). Recent advances in antiinfective therapy against lung pathogens have dramatically contributed to increased life expectancy of CF patients. However, frequent and prolonged antibiotic courses are likely to be a major factor in the selection of highly antibiotic-resistant P. aeruginosa strains. Similar resistance issues have arisen for human fungal pathogens. The resistance problems are enhanced in HIV patients and other individuals with compromised immune systems due to chemotherapy, organ transplants, and long-term hospitalization (M A. Ghannoum and L B. Rice. 1999. Antifungal Agents Mode of Action, Mechanisms of Resistance, and Correlation of These Mechanisms with Bacterial Resistance. Clin. Microbiol. Rev. 12:501-517).

A viral infection begins when a virion comes into contact with a host cell and attaches or adsorbs, to it. The viral (DNA or RNA) then crosses the plasma membrane into the cytoplasm and eventually enter into the nucleus. In the case of retrovirus, the viral RNA is reverse transcribed into DNA. Viral DNA is then integrated into the chromosomal DNA of the infected cell. Integration is mediated by an integration protein, integrase. All integrated proviruses are required for the subsequent transcription process which is acted upon by the host cell transcription factors. The integrated DNA is transcribed by the cell's own machinery into mRNA, or replicated and becomes enclosed in a virion. For retrovirus, the integrated DNA is transcribed into RNA that either acts as mRNA or become enclosed in a virion. This completes the virus life cycle.

Seasonal waves of influenza virus infections have caused over 36,000 deaths per year in the United States alone (Smith N M, Bresee J S, Shay D K, Uyeki T M, Cox N J, Strikas R A: Center for Disease Control and Prevention: Prevention and control of influenza: recommendations of the Advisory Committee on Immunization Practices (APIP). MMWR Recomm Rep 2006, 55:1-42). Less than 100 years ago, a single strain of H1N1 influenza virus caused a pandemic with more human fatalities than any other single infectious event, war, or famine in world history (Achievements in Public Health, 1900-1999: Control of Infectious Diseases. MMWR Morb Mortal Wkly Rep 1999, 48:621-629). More recently, a highly pathogenic H5N1 strain of avian influenza has been repeatedly transmitted from birds to humans, resulting in several hundred human deaths (Update: influenza activity—United States and worldwide, 2005-06 season, and composition of the 2006-07 influenza vaccine. MMWR Morb Mortal Wkly Rep 2006, 55:648-653; World Heath Organization: H5N1 avian influenza: timeline of major events [http://www.who.int/csr/disease/avian_influenza/timeline_{—}2007_{—}05_{—}10.pdf]). Fortunately, this has generated few cases of human-to-human transmission and has not yet resulted in a major human pandemic (Webster R G, Peiris M, Chen H, Guan Y: H5N1 outbreaks and enzootic influenza. Emerg Inf Dis 2006, 12:3-8; Nicholls J M, Chan M C W, Chan W Y, Wong H K, Cheung, C Y, Kwong L W, Wong M P, Chui, W H, Poon L L M, Tsao S W, Guan Y, Peiris, JSM: Tropism of avian influenza A (H5N1) in the upper and lower respiratory tract. Nature Med 2007, 13: 147-149). It is clear that the natural influenza reservoir has the capacity to generate new virus strains that can cross species barriers and produce human infections with increased pathogenicity and in some cases increased human-to-human transmission characteristics. These strains present a real and potentially uncontrollable threat to global public health (Nelson, M I, Holmes, E C: The evolution of epidemic influenza. Nat Rev Genetics 2007, 8:196-205).

Influenza viruses are lipid enveloped, with segmented, negative-stranded RNA genomes. (Webster R G, Bean W J, Gorman O T, Chambers T M, Kawaoka Y: Evolution and ecology of influenza A viruses. Microbiol. Rev 1992, 56:152-179; Wright P F, Webster R G: Orthomyxoviruses. In Fields Virology, 4th edition. Edited by Fields B N, Knipe D M, Howley P M. Philadelphia: Lippincott Williams & Wilkins; 2001:1534-1579). They are capable of rapid evolution through the accumulation of point mutations as well as by re-assortment of RNA segments to generate novel progeny (Wright P F, Webster R G: Orthomyxoviruses. In Fields Virology, 4th edition. Edited by Fields B N, Knipe D M, Howley P M. Philadelphia: Lippincott Williams & Wilkins; 2001:1534-1579). The ecological cycles of influenza viruses include replication in a large and genetically diverse wild reservoir dominated by water birds as hosts (Wright P F, Webster R G: Orthomyxoviruses. In Fields Virology, 4th edition. Edited by Fields B N, Knipe D M, Howley P M. Philadelphia: Lippincott Williams & Wilkins; 2001:1534-1579). Viruses from this reservoir continually spill over into other avian and mammalian host populations, including humans (Hlinak A, Mühle R U, Werner O, Globig A, Starick E, Schirrmeier H, Hoffmann B, Engelhardt A, Hübner D, Conraths F J, Wallschläger D, Kruckenberg H, Müller T: A virological survey in migrating waders and other waterfowl in one of the most important resting sites of Germany. J Vet Med B Infect Dis Vet Public Health 2006, 53:105-110; Humberd J, Guan Y, Webster R G: Comparison of the replication of influenza A viruses in Chinese ring-necked pheasants and Chukar partridges. J Virol 2006, 80:2151-61; Perdue M L, Swayne D E: Public health risk from avian influenza viruses. Avian Dis 2005, 49: 317-327; Alexander D J, Brown I H: Recent zoonoses caused by influenza A viruses. Rev Sci Tech 2000, 19: 197-225). Survivors of influenza virus infection generally mount an immune response with only limited cross-reactivity to other influenza strains, resulting in multiple infections throughout an individual's life time (Couch R B: An overview of serum antibody responses to influenza virus antigens. Dev Biol (Basel) 2003, 115:25-30), and multiple epidemics and pandemics (Kilbourne E D: Influenza pandemics of the 20th century. Emerg Infect Dis 2006, 12:9-14) when previously exposed populations are confronted with new virus strains (Alexander D J, Brown I H: Recent zoonoses caused by influenza A viruses. Rev Sci Tech 2000, 19:197-225; Influenza vaccines. Wkly Epidemiol Rec 2005, 80:277-288; Webster R G: Immunity to influenza in the elderly. Vaccine 2000, 18:1686-1689).

Current influenza control efforts have concentrated on the use of vaccines and a small number of anti-influenza drugs. Because influenza vaccines are only partially cross-protective, they must be developed and produced de novo each year, based on predictions of which strains are likely to circulate the following year (Recommended composition of influenza virus vaccines for use in the 2007 influenza season. Wkly Epidemiol Rec 2006, 81:390-395). This prevents stockpiling and use of vaccination distribution strategies to control future severe outbreaks. Two main classes of anti-influenza drugs have been developed and are in current use. Inhibitors of the viral ion channel M protein, such as amantidine (Davies W L, Grunert R R, Haff R F, McGahen J W, Neumayer E M, Paulshock M, Watts J C, Wood R T, Hermann E C, Hoffmann C E: Antiviral activity of 1-adamantanamine (amantadine). Science 1964, 144:862-863; Shimbo K, Brassard D L, Lamb R A, Pinto L H: Ion selectivity and activation of the M2 ion channel of influenza virus. Biophys J 1996, 70:1335-1346) and rimantidine (Rabinovich S, Baldini J T, Bannister R: Treatment of influenza. The therapeutic efficacy of rimantidine HC1 in a naturally occurring influenza A2 outbreak. Am J Med Sci 1969, 257:328-335; Chizhmakov I V, Geraghty F M, Ogden D C, Hayhurst A, Antoniou M, Hay A J: Selective proton permeability and pH regulation of the influenza virus M2 channel expressed in mouse erythroleukaemia cells. J Physiol pt 2 1996, 494:329-336), have been produced and commercialized, as well as have inhibitors of the viral surface neuraminidase enzyme, such as oseltamivir (Kati W M, Saldivar A S, Mohamadi F, Sham H L, Layer W G, Kohlbrenner W E: GS4071 is a slow-binding inhibitor of influenza neuraminidase from both A and B strains which is now in wide use. Biochem Biophys Res Commun 1998, 244:408-413),. These drugs are effective as prophylactics in blocking the development of influenza virus symptoms (Parker R, Loewen N, Skowronski D: Experience with oseltamivir in the control of a nursing home influenza B outbreak. Can Commun Dis Rep 2001, 27:37-40) as well as therapeutically treating (Kawai N, Ikematsu H, Iwaki N, Maeda T, Satoh I, Hirotsu N, Kashiwagi S: A Comparison of the effectiveness of oseltamivir for the treatment of influenza A and influenza B: A Japanese multicenter study of the 2003-2004 and 2004-2005 influenza seasons. Clin Infect Dis 2006, 43:439-444), and reducing the duration of symptoms post-infection (Gillissen A, Höffken G: Early therapy with the neuraminidase inhibitor oseltamivir maximizes its efficacy in influenza treatment. Med Microbiol Immunol 2002, 191:165-168). Nevertheless, due to the ability of influenza viruses to rapidly mutate, drug resistance against each of the antiviral classes has appeared quickly (Smith N M, Bresee J S, Shay D K, Uyeki T M, Cox N J, Strikas R A: Center for Disease Control and Prevention: Prevention and control of influenza: recommendations of the Advisory Committee on Immunization Practices (APIP). MMWR Recomm Rep 2006, 55:1-42; Moscona A: Neuraminidase inhibitors for influenza. New Engl J Med 2005, 353:1363-137327). Today, the M protein inhibitors, amantidine and rimantidine, are no longer in common use in many areas because of viral resistance, just a few years after their commercial distribution (Saito R, Sakai T, Sato I, Sano Y, Oshitani H, Sato M, Suzuki H: Frequency of amantadine-resistant influenza A viruses during two seasons featuring co-circulation of H1N1 and H3N2. J Clin Microbiol 2003, 41:2164-2165). Resistance to oseltamivir has also been reported in human and avian influenza viruses, and is predicted to increase with increased usage (Guberava L V, Kaiser L, Matrosovich M N, Soo-Hoo Y, Hayden F G: Selection of influenza virus mutants in experimentally infected volunteers treated with oseltamivir. J Infect Dis 2001, 183:523-531; Kiso M, Mitamura K, Sakai-Tagawa Y, Shiraishi K, Kawakami C, Kimura K, Hayden F G, Sugaya N, Kawaoka Y: Resistant influenza A viruses in children treated with oseltamivir: descriptive study. Lancet 2004, 364:759-765). New anti-influenza drugs will be required to keep pace with the ability of influenza viruses to mutate and develop resistance to current drugs.

The discovery of AZT as an effective disrupter of the HIV-1 viral cycle has improved the quality of life and extended the lives of many HIV positive individuals, though often with significant side effects. Unfortunately, regular use of AZT and other viral reverse transcriptase inhibitors, HIV proteases inhibitors, and Highly Active Antiretroviral Therapy (HAART) that involves multi-drug therapies has lead to the generation of resistant HIV strains, making control of HIV viral load in HIV+ and AIDS patients more difficult. The development of enfuvirtide (also termed T-20 or Fuzeon®), which controls HIV strains resistant to nucleosides, non-nucleosides, nucleotides, and protease inhibitors, through blocking viral fusion, was a significant advancement in HIV treatments because it addressed a new therapeutic target. Although very effective, there are major drawbacks that limit its compliance and use in non-clinical settings. The need for new HIV therapies that have novel therapeutic targets is well recognized and is an imperative for this global public health problem.

In the past two decades, the emergence of human immunodeficiency virus type 1 (HIV-1), Human Influenza (H1N1), Avian Flu (H5N1), Dengue, and West Nile virus as an important human pathogens has led to a resurgence of scientific interest in retroviruses and other viruses. Unfortunately, for viruses like Dengue, there are no known treatments and the numbers of cases worldwide are increasing dramatically, with significant northern latitude expansion of infection due to the northern drift of the Aedes aegypti mosquito, the insect host for Dengue viruses. Current antivirals target, for the most part, various steps in the viral replication cycle, and resistance to these agents is significant, particularly with patients with HIV-1 infections (Pillay D. 1998. Emergence and control of resistance to antiviral drugs in resistance in herpes viruses, hepatitis B virus, and HIV. Commun Dis Public Health 1:5-13; Larder B A. 1996. Nucleoside and foscarnet-mechanisms. In: Richman D D, ed. Antiviral Drug Resistance. London: Wiley, pp. 169-190).

Prions, or proteinaceous infectious particles, are the cause of a number transmissible of neurodegenerative diseases in mammals that include bovine spongiform encephalopathies (BSE) (Westaway, D, Telling, G. and Priola, S. 1998. Prions. Proc. Natl. acad. Sci. USA 95:11030-11031. In the mid 1980's, over 200,000 cases of BSE were reported, though human cases are much lower. Belay, E. D. and Schonberger, L. B. 2005. The Public health impact of Prion diseases. Annu. Rev. Public Health 26:191-212). Prions are malformed proteins that form plaques or amyloids on cerebral neuronal tissues leading to disruption of neuron function and apoptosis. Amyloids is a general term for protein fragments that the body produces normally, and in the case of prions, the amyloids are proteins with an aberrant folding or conformation. There are no current treatments for these progressive disorders or drugs that prevent amyloid generation and deposition.

Pathogens, bacterial, viral fungal and protozoan, have very serious impacts on animal health ranging from wild species and livestock to domesticated pets. Many viral and bacterial based diseases can devastate natural populations and severely influence agricultural production. These include a broad range of influenza viruses that are selective for fowl or porcine, foot-and-mouth disease viruses (FMDV) that are the prototypic member of the Aphthovirus genus in the Picornaviridae family. This picornavirus is the etiological agent of the acute systemic vesicular disease that affects cattle and other animals worldwide. It is a highly variable and transmissible virus that is a highly contagious, and sometimes fatal, viral disease of cattle and pigs. It can also infect deer, elk, antelope, bison, water buffalo, goats, sheep, and other bovids with cloven hooves. Fowl Pox viruses are very serious as well as avian flu viruses (Highly pathogenic Bird flu, H5N1). A range of bacterial pathogens exists, from those that can cause death in the host to those that are more pathogenic to humans if infected animals are consumed. Salmonella spp. infections are common in processing plants, but are GI pathogens in chickens and turkeys. Coliform bacterial species that infect the gut can have huge impacts on product and outbreaks in Asia have required destruction of 70-80% of the animal crop in any given year. In particular, enterohaemorrhagic forms of the bacterium E. coli have had devastating impacts on animal production. Therefore effective and human-safe treatments and prophylactics for animal-based pathogens, including vaccines, are critical. Several effective anti-virals and anti-bacterials have been banned because there use has resulted in a high degree of pathogen resistance.

Plants are constantly challenged by a wide variety of pathogenic organisms including fungi, viruses, and bacteria. Attempts have been made to control plant disease by means of disinfections, replacement of the soil, various cultural practices, genetic engineering of the plant, and control by chemicals. Some plants suffer from detrimental soil-spread diseases, which have not been possible to control owing to restrictions of use of chemical control agents and hazard periods due to possible residues or lack of sufficiently effective products. Extensive use of a broad range of anti-fungal agents on crops has lead to increasing rates of resistance, and current resistance to potato blight and soybean rust pathogens may have significant impacts of global food production (Eds. H. Lyr, P. E. Russell & H. D. Sisler. 1996. Modern fungicides and antifungal compounds. Intercept Ltd, Andover, Hants, 578 pp).

Protozoa and related eukaryotic parasites are major causes of disease including malaria, Giardia and other water-borne protozoans, certain sexually transmitted diseases, sleeping sickness (Trypanosomiasis), Leishmania, and a host of worm parasites (Quellette, M. 2001. Biochemical and molecular mechanisms of drug resistance in parasites. Trop. Med. Internatl. Health 60:874-882; White, N J. 2004. Anti-malarial drug resistance. J. Clin. Internatl. 110: 1084-1092). It has been estimated that at least one-third of the world's human population is threatened by protozoan parasites. Resistance to such anti-protozoan drugs such as the sulfonamides, Chloroquine, Benimadazole, and Ivermectin is found worldwide and rates of resistance are increasing at an alarming rate. New drug targets, modes-of-action, and combination of drugs for anti-protozoan drugs are desperately needed that can not only overcome rapid resistance generation, but that minimize side effects and are cost effective.

Historically, a wide variety of medicinals for the treatment and prevention of infectious diseases have been derived from plants, and plants continue to be a major source of novel compounds for drug development. Among many others, this includes shikimic acid, the starting compound for oseltamivir synthesis, and the anti-malarial, artemisin (qinghaosu) (Abrecht S, Harrington P, Iding H, Karpf M, Trussardi R, Wirz B, Zutter U: The synthetic development of the anti-influenza neuraminidase inhibitor oseltamivir phosphate (Tamiflu®): A challenge for synthesis & process research. Chimia 2004, 58:621-629; Qinghaosu Antimalarial Coordinating Research Group Antimalarial studies on qinghaosu. Chin Med J 1979, 92: 811-816). The phytochemical literature contains multiple reports of anti-influenza properties of extracts from plant species including elder berry (Sambucus nigra L.) (Serkedjieva, J, Manolova, N, Zgorniak-Nowosielska, I, Zawilinska, B, Grzybek, J: Antiviral activity of the infusion (SHS-174) from flowers of Sambucus nigra L., aerial parts of Hypericum perforatum L., and roots of Saponaria officinilis L. against influenza and herpes simplex viruses. Phytother Res 1990, 4:97-100; Zakay-Rones Z, Varsano N, Zlotnik M, Manor O, Regev L, Schlesinger M, Mumcuoglu M: Inhibition of several strains of influenza virus in vitro and reduction of symptoms by an elderberry extract (Sambucus nigra L.) during an outbreak of influenza B Panama. J Altern Complement Med 1995, 1:361-369; Burge E, Mumcuoglu M, Simmons T: The effect of Sambucol on flu-like symptoms in chimpanzees: prophylactic and symptom-dependent treatment. Int Zoo News 1999, 46:16-19; Zakay-Rones Z, Thom E, Wollan T, Wadstein J: Randomized study of the efficacy and safety of oral elderberry extract in the treatment of influenza A and B virus infections. J Internatl Med Res 2004, 32:132-140), green tea (Camellia sinensis) (Song J-M, Lee K-H, Seong B-L: Antiviral effect of catechins in green tea on influenza virus. Antiviral Res 2005, 68:66-74; Imanishi N, Tuji Y, Katada Y, Maruhashi M, Konosu S, Mantani N, Terasawa K, Ochiai H: Additional inhibitory effect of tea extract on the growth of influenza A and B viruses in MDCK cells. Microbiol Immunol 2002, 46:491-494), geranium (Geranium sanguineum L.) (Serkedjieva J A, Hay A: In vitro antiinfluenza virus activity of a plant preparation from Geranium sanguineum L. Antiviral Res 1998, 37:221-230; Serkedjieva J: Influenza virus variants with reduced susceptibility to inhibition by a polyphenol extract from Geranium sanguineum L. Pharmazie 2003, 58:53-57; Sokmen M, Angelova M, Krumova E, Pashov S, Ivanchev S, Sokmen A, Serkedjieva J: In vitro antioxidant activity of polyphenol extracts with antiviral properties from Geranium sanguineum L. Life Sci 2005, 76:2981-2993; Pantev A, Ivancheva S, Staneva L, Serkedjieva J: Biologically active constituents of a polyphenol extract from Geranium sanguineum L. with anti-influenza activity. Z Naturforsch [C] 2006, 61:508-516), black currant (Ribes nigrum L.) (Knox Y M, Hayashi K, Suzutani T, Ogasawara M, Yoshida I, Shiina R, Tsukui A, Terahara N, Azuma M: Activity of anthocyanins from fruit extract of Ribes nigrum L. against influenza A and B viruses. Acta Virol 2001, 45:209-215; Knox Y M, Suzutani T, Yosida I, Azuma M: Anti-influenza virus activity of crude extract of Ribes nigrum L. Phytother Res 2003, 17:120-122), buckeye (Aesculus chinensis Bge.) (Wei F, Ma S—C, Ma L-Y, But P P-H, Lin R-C, Khan I A: Antiviral flavonoids from the seeds of Aesculus chinensis. J Nat Prod 2004, 67: 650-653), and greater grasshopper tree (Pithecellobium clypearia (Jack) Benth). Li Y, Leung K-T, Yao F, Ooi L S M, Ooi V E C: Antiviral flavans from the leaves of Pithecellobium clypearia. J Nat Prod 2006, 69:833-835. Elder berry, in particular, has been widely utilized for treating upper respiratory maladies, with documentation for this use dating from Hippocrates in the 5^{th} century B.C. Moreover, three studies have documented the effectiveness of elder berry extracts in treating influenza infections in chimpanzees and humans (Zakay-Rones Z, Varsano N, Zlotnik M, Manor O, Regev L, Schlesinger M, Mumcuoglu M: Inhibition of several strains of influenza virus in vitro and reduction of symptoms by an elderberry extract (Sambucus nigra L.) during an outbreak of influenza B Panama. J Altern Complement Med 1995, 1:361-369; Burge E, Mumcuoglu M, Simmons T: The effect of Sambucol on flu-like symptoms in chimpanzees: prophylactic and symptom-dependent treatment. Int Zoo News 1999, 46:16-19; Zakay-Rones Z, Thom E, Wollan T, Wadstein J: Randomized study of the efficacy and safety of oral elder berry extract in the treatment of influenza A and B virus infections. J Internatl Med Res 2004, 32:132-140). However, a major problem in understanding, comparing and utilizing chemically complex extracts from botanicals lies in the variability of the plant sources and methods of preparation. In particular, different studies of elder berry anti-influenza activity have used extracts from either flowers or fruits, prepared in different ways, and either with or without additives (Serkedjieva, J, Manolova, N, Zgorniak-Nowosielska, I, Zawilinska, B, Grzybek, J: Antiviral activity of the infusion (SHS-174) from flowers of Sambucus nigra L., aerial parts of Hypericum perforatum L., and roots of Saponaria officinilis L. against influenza and herpes simplex viruses. Phytother Res 1990, 4:97-100; J Altern Complement Med 1995, 1:361-369; Burge E, Mumcuoglu M, Simmons T: The effect of Sambucol on flu-like symptoms in chimpanzees: prophylactic and symptom-dependent treatment. Int Zoo News 1999, 46:16-19; Zakay-Rones Z, Thom E, Wollan T, Wadstein J: Randomized study of the efficacy and safety of oral elderberry extract in the treatment of influenza A and B virus infections. J Internatl Med Res 2004, 32:132-140).

The present invention provides in part improved antiinfective agents based on identified bioactives that have demonstrated antiinfective activity.

### SUMMARY

One aspect of the invention relates to pure and isolated esterified flavononols represented by formula I:

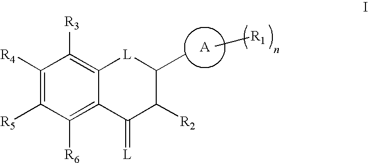

wherein, independently for each occurrence:

R₁ represents alkoxy, alkenyloxy, alkynyloxy, aryloxy, arylalkyloxy, hydroxy, —OC(O)—R₇, alkyl, alkenyl, alkynyl, acetyl, formyl, halide, cyano, nitro, SH, amino, amido, sulfonyl, or sulfonamido;

R₂ represents —OH or

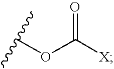

R₃, R₄, R₅, and R₆ represent H, hydroxy, alkoxy, alkenyloxy, alkynyloxy, aryloxy, aralkyloxy; —OC(O)—R₇, alkyl, alkenyl, alkynyl, aralkyl, acetyl, formyl, halide, cyano, nitro, SH, amino, amido, sulfonyl, or sulfonamido;

R₇ represents H, alkyl, alkenyl, alkynyl, cycloalkyl, aryl, arylalkyl or a carbohydrate;

A represents an aryl group;

L represents O, S, or NR;

R represents H, hydroxy, alkyl, alkenyl, alkynyl, aralkyl, acetyl, formyl, or sulfonyl;

X represents a carbohydrate, a cycloalkyl, or a cycloalkenyl group; and

n represents an integer from 1 to 5, inclusive;

wherein any of the aforementioned alkoxy, alkenyloxy, alkynyloxy, aryloxy, aralkyloxy, alkyl, alkenyl, alkynyl, cycloalkyl, cycloalkenyl, aryl and aralkyl groups may be optionally substituted with one or more groups selected from the group consisting of hydroxy, alkoxy, alkenyloxy, alkynyloxy, aryloxy, aralkyloxy; halide, formyl, acetyl, cyano, nitro, SH, amino, amido, sulfonyl, or sulfonamido.

In another aspect, the present invention relates to a pharmaceutical composition comprising a compound of the present invention and a pharmaceutically acceptable carrier.

In another aspect, the present invention relates to a method of treating a subject for an infection comprising administering to the subject in need thereof and effective amount of a compound of the present invention. In a further embodiment, the infection is a viral, bacterial, fungal, or prion infection.

Another aspect of the invention relates to a vaccine derived from a influenza viral ‘adhesin’ domain that is the 3-7 amino acid binding site of compounds of the present invention. In a further embodiment the binding sequences are used as antigens for vaccine production and such resulting vaccine would have broad anti-influenza activity.

In another aspect, the present invention relates to a method of detecting a microbial agent with a pharmaceutical composition of the present invention.

In another aspect, the present invention relates to the methods of making through extraction and purification from natural sources pharmaceutical compositions of the present invention. In certain embodiments, the sources include but are not restricted to elder berry (Sambucus nigra L.) fruits and flowers, green tea (Camellia sinensis) leaves, cinnamon bark (Cinnamomum cassia), pine bark (Pinus marita, P. radiata), cherries (Prunus spinosa, Prunus spp.), cranberry fruits (Vaccinium macrocarpon), and persimmon (Diospyro virginiana, D. kaki, D. digna, D bicolor, D. loutus).

In another aspect, the present invention relates to the methods of making the pharmaceutical compositions of the present invention using methods known in the synthetic organic chemistry art.

These embodiments of the present invention, other embodiments, and their features and characteristics, will be apparent from the description, drawings and claims that follow.

### BRIEF DESCRIPTION OF DRAWINGS

FIG. 1 depicts the DART TOF-MS positive ion mass spectrum of compounds of the present invention that bind to the H1N1 virus surface after a 1-hr incubation in an elder berry extract. The peak at m/z=479.51 represents an esterified flavononol, termed Tristenonol, while the peak at m/z=214.09 represents a DART TOF-MS generated fragment of Tristenonol.

FIG. 2 depicts the DART TOF-MS positive ion mass spectrum of compounds of the present invention that bind to the H5N1 virus surface after a 1-hr incubation in an elder berry extract. The peak at m/z=479.51 represents an esterified flavononol, termed Tristenonol, while the peak at m/z=214.09 represents a DART TOF-MS generated fragment of Tristenonol.

FIG. 3 depicts the DART TOF-MS positive ion mass spectrum of a compound of the present invention bound to the H1N1 virus surface after a 1-hr incubation in the presence of the synthesized flavononol, the Tristenonol aglycone.

FIG. 4 depicts the structures for the esterified flavononol esterified or glycosylated on the C ring with shikimic acid that uniquely binds to H1N1 and H5N1 virions and not other enveloped or non-envelope viruses.

FIG. 5 depicts a comparison on the 2-D (A) and 3-D (B) structures of the novel esterified flavonolol compounds of the present invention that uniquely binds to influenza viruses.

FIG. 6 depicts an extraction and purification scheme from a botanical for the 479.5 m/z flavononols and leucoanthocyanidins. A botanical extract (powder or paste) is extracted with warm water (40 C) and the eluate is loaded onto Celite 545 and the pellet is discarded. The strating extract can also be loaded on LH20 and fractions collected. The celite bound material is washed with low ionic strength Tris-HCl buffer (pH 8.2), and the washed material discarded. The Celite-bound fraction is released with high ionic strength K-phosphate buffer and collected, then loaded onto hydroylapatite. The fractions of interest, f esterified flavononol or the aglycones are collected with an increasing gradient of K-phosphate buffer, and the lower molecular weight (<250 MW) phenolic fraction is discarded.

FIG. 7 depicts the tethered form of the pharmaceutical compositions as used for detection, identification, decontamination and protection from infectious bacterial, fungal, viral and prion agents and non-infectious amyloid agents. The chemical tether, either an ester or amide linkage to the A ring of the monomer of the pharmaceutical compositions here are shown as Δ. The tether is preferred on the A ring so that the active binding domain defined by the two phenolic rings of Rings B and C are free to interact with binding motifs on the targeted pathogens

FIG. 8 depicts the solution form of the pharmaceutical compositions as used for detection, identification, decontamination and protection from bacterial, fungal, and viral infections. The active phenolic binding domains of Rings B and C of the pharmaceutical compositions here interact with binding motifs on the targeted pathogens.

FIG. 9 depicts a device for detection/identification of infectious agents and amyloid agents in an aqueous environment or vapor phase environment. The device include a means of collected the sample stream, interrogating that stream with a solid support film on which the pharmaceutical compositions here are tethered and available for binding targeted ligands—pathogens or amyloids, and for which the binding event reports the detection/identification of said target through an optical or other physical signal that reports the recognition event.

FIG. 10 depicts the Direct Binding Assay wherein a pure compound of the present invention or a botanical extract is used to identify chemistry present in a mixture that bind to target pathogens or amyloids by incubating a pathogen or amyloid fraction in said pure compound of the present invention or botanical extract and then using the DART TOF-MS to determine the mass and identity of pathogen or amyloid surface bound compounds.

### DETAILED DESCRIPTION OF THE INVENTION

For convenience, before further description of the disclosure, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The term “acyl” as used herein refers to the radical

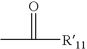

wherein R′₁₁ represents hydrogen, alkyl, alkenyl, alkynyl, or —(CH₂)ₘ—R₈₀, wherein R₈₀ is aryl, cycloalkyl, cycloalkenyl, heteroaryl or heterocyclyl; and m is an integer in the range 0 to 8, inclusive.

The term “alkyl” refers to a radical of a saturated straight or branched chain hydrocarbon group of, for example, 1-20 carbon atoms, or 1-12, 1-10, or 1-6 carbon atoms.

The term “alkenyl” refers to a radical of an unsaturated straight or branched chain hydrocarbon group of, for example, 2-20 carbon atoms, or 2-12, 2-10, or 2-6 carbon atoms, having at least one carbon-carbon double bond.

The term “alkynyl” refers to a radical of an unsaturated straight or branched chain hydrocarbon group of, for example, 2-20 carbon atoms, or 2-12, 2-10, or 2-6 carbon atoms, having at least one carbon-carbon triple bond.

The term “aliphatic” includes linear, branched, and cyclic alkanes, alkenes, or alkynes. In certain embodiments, aliphatic groups in the present invention are linear, branched or cyclic and have from 1 to about 20 carbon atoms.

The term “aralkyl” includes alkyl groups substituted with an aryl group or a heteroaryl group.

The term “heteroatom” includes an atom of any element other than carbon or hydrogen. Illustrative heteroatoms include boron, nitrogen, oxygen, phosphorus, sulfur and selenium, and alternatively oxygen, nitrogen or sulfur.

The term “halo” or “halogen” includes —F, —Cl, —Br, — or —I.

The term “perfluoro” refers to a hydrocarbon wherein all of the hydrogen atoms have been replaced with fluorine atoms. For example, —CF₃ is a perfluorinated methyl group.

The term “aryl” refers to a mono-, bi-, or other multi-carbocyclic, aromatic ring system. The aryl group can optionally be fused to one or more rings selected from aryls, cycloalkyls, and heterocyclyls. The aryl groups of this invention can be substituted with groups selected from alkyl, alkenyl, alkynyl, alkanoyl, alkoxy, alkoxy, alkylthio, amino, amido, aryl, aralkyl, azide, carbonyl, carboxy, cyano, cycloalkyl, ester, ether, halogen, haloalkyl, heterocyclyl, hydroxy, imino, ketone, nitro, perfluoroalkyl, phosphonate, phosphinate, silyl ether, sulfonamido, sulfonate, sulfonyl, and sulfhydryl.

The term “heteroaryl” refers to a mono-, bi-, or multi-cyclic, aromatic ring system containing one, two, or three heteroatoms such as nitrogen, oxygen, and sulfur. Examples include pyrrole, furan, thiophene, imidazole, oxazole, thiazole, triazole, pyrazole, pyridine, pyrazine, pyridazine and pyrimidine, and the like. Heteroaryls can also be fused to non-aromatic rings.

The terms “heterocycle,” “heterocyclyl,” or “heterocyclic” refer to a saturated or unsaturated 3-, 4-, 5-, 6- or 7-membered ring containing one, two, or three heteroatoms independently selected from nitrogen, oxygen, and sulfur. Heterocycles can be aromatic (heteroaryls) or non-aromatic. Heterocycles can be substituted with one or more substituents including alkyl, alkenyl, alkynyl, aldehyde, alkylthio, alkanoyl, alkoxy, alkoxycarbonyl, amido, amino, aminothiocarbonyl, aryl, arylcarbonyl, arylthio, carboxy, cyano, cycloalkyl, cycloalkylcarbonyl, ester, ether, halogen, heterocyclyl, heterocyclylcarbonyl, hydroxy, ketone, oxo, nitro, sulfonate, sulfonyl, and thiol.

Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl. Heterocycles also include bridged bicyclic groups where a monocyclic heterocyclic group can be bridged by an alkylene group.

The heterocyclic or heteroaryl ring may be can be substituted with groups selected from alkyl, alkenyl, alkynyl, alkanoyl, alkoxy, alkoxy, alkylthio, amino, amido, aryl, aralkyl, azide, carbonyl, carboxy, cyano, cycloalkyl, ester, ether, halogen, haloalkyl, heterocyclyl, hydroxy, imino, ketone, nitro, perfluoroalkyl, phosphonate, phosphinate, silyl ether, sulfonamido, sulfonate, sulfonyl, and sulfhydryl.

The terms “polycyclyl” and “polycyclic group” include structures with two or more rings (e.g., cycloalkyls, cycloalkenyls, cycloalkynyls, aryls and/or heterocyclyls) in which two or more carbons are common to two adjoining rings, e.g., the rings are “fused rings.” Rings that are joined through non-adjacent atoms, e.g., three or more atoms are common to both rings, are termed “bridged” rings. Each of the rings of the polycycle may be substituted with such substituents as described above can be substituted with groups selected from alkyl, alkenyl, alkynyl, alkanoyl, alkoxy, alkoxy, alkylthio, amino, amido, aryl, aralkyl, azide, carbonyl, carboxy, cyano, cycloalkyl, ester, ether, halogen, haloalkyl, heterocyclyl, hydroxy, imino, ketone, nitro, perfluoroalkyl, phosphonate, phosphinate, silyl ether, sulfonamido, sulfonate, sulfonyl, and sulfhydryl.

The term “carbocycle” includes an aromatic or non-aromatic ring in which each atom of the ring is carbon.

The terms “amine” and “amino” include both unsubstituted and substituted amines, e.g., a moiety that may be represented by the general formulas:

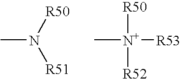

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and includes a moiety that may be represented by the general formula:

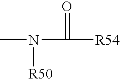

wherein R50 is as defined above, and R54 represents a hydrogen, an alkyl, an alkenyl or —(CH₂)ₘ—R61, where m and R61 are as defined above.

The term “amido” refers to an amino-substituted carbonyl and includes a moiety that may be represented by the general formula:

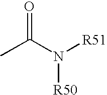

wherein R50 and R51 are as defined above. Certain embodiments of the amide in the present invention will not include imides which may be unstable.

The term “alkylthio” includes an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” includes such moieties as may be represented by the general formulas:

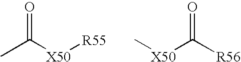

wherein X50 is a bond or represents an oxygen or a sulfur, and R55 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiocarbonyl” group. Where X50 is a sulfur and R55 or R56 is not hydrogen, the formula represents a “thioester.” Where X50 is a sulfur and R55 is hydrogen, the formula represents a “thiocarboxylic acid.” Where X50 is a sulfur and R56 is hydrogen, the formula represents a “thioformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” include an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” includes a moiety that may be represented by the general formula:

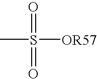

in which R57 is an electron pair, hydrogen, alkyl, cycloalkyl, or aryl.

The term “sulfate” includes a moiety that may be represented by the general formula:

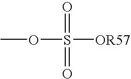

in which R57 is as defined above.

The term “sulfonamido” is art-recognized and includes a moiety that may be represented by the general formula:

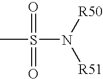

in which R50 and R51 are as defined above.

The term “sulfonyl” includes a moiety that may be represented by the general formula:

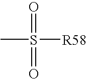

in which R58 is one of the following: hydrogen, alkyl, alkenyl, alkynyl, cycloalkyl, heterocyclyl, aryl or heteroaryl.

The term “sulfoxido” includes a moiety that may be represented by the general formula:

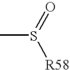

in which R58 is defined above.

The term “optionally substituted” or “substituted” is contemplated to include all permissible substituents of organic compounds. For example, substituted refers to a chemical group, such as alkyl, cycloalkyl, aryl, heteroaryl and the like, wherein one or more hydrogen atoms may be replaced with a substituent such as halogen, azide, alkyl, aralkyl, alkenyl, alklynyl, cycloalkyl, hydroxy, alkoxy, amino, amido, nitro, cyano, sulfhydryl, imino, phosphonate, phosphinate, carbonyl, carboxyl, silyl, ether, alkylthio, sulfonyl, sulfonamido, ketone, aldehyde, ester, heterocyclyl, aromatic or heteroaromatic moieties, perfluoroalkyl (e.g. —CF₃), acyl, and the like, or any of the substituents of the preceding paragraphs or any of those substituents either attached directly or by suitable linkers. The linkers are typically short chains of 1-3 atoms containing any combination of —C—, —C(O)—, —NH—, —S—, —S(O)—, —O—, —C(O)O— or —S(O)—. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms.

The definition of each expression, e.g. alkyl, m, n, etc., when it occurs more than once in any structure, is intended to be independent of its definition elsewhere in the same structure unless otherwise indicated expressly or by the context.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms are art recognized and represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

The term “hydrocarbon” includes all permissible compounds having at least one hydrogen and one carbon atom. For example, permissible hydrocarbons include acyclic and cyclic, branched and unbranched, carbocyclic and heterocyclic, aromatic and nonaromatic organic compounds that may be substituted or unsubstituted.

The phrase “protecting group” includes temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed. Greene et al., Protective Groups in Organic Synthesis 2^{nd} ed., Wiley, New York, (1991). The phrase “hydroxyl-protecting group” includes those groups intended to protect a hydroxyl group against undesirable reactions during synthetic procedures and includes, for example, benzyl or other suitable esters or ethers groups known in the art. The aforementioned protecting groups may be present in the compounds of the invention, and are not limited to use only during synthesis of the compounds of the invention. Thus, the presence of a protecting group is not intended to suggest that said group must be removed. For example, the compounds of the present invention may contain an ether group, such as a methoxymethyl ether, which is a known hydroxyl protecting group.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

The term “effective amount” as used herein refers to the amount necessary to elicit the desired biological response. As will be appreciated by those of ordinary skill in this art, the effective amount of a drug may vary depending on such factors as the desired biological endpoint, the drug to be delivered, the composition of any additional active or inactive ingredients, the target tissue, etc.

The term “vaccine” as used herein refers to a proteinaceous antigen produced by the immune system after being introduced into a vertebrate system that recognizes specific surface recognition elements on target pathogens and targets them for removal/destruction by specific immune cells like leucocytes and macrophages. In the case of influenza viruses, such vaccines are very strain-specific.

As used herein, the term “envelope virus” refers to a virus comprising a lipid bilayer containing viral glycoproteins derived from a host cell membrane. In an envelope virus, viral proteins that mediate attachment and penetration into the host cell are found in the envelope. Examples of envelope viruses include influenza, both human and avian, human immunodeficiency virus (HIV), (sudden acute respiratory syndrome (SARS), human papilloma virus (HPV), herpes simplex virus (HSV), Dengue and other flavie viruses, such as for example, Yellow Fever, West Nile, and Encephalitis viruses.

A “flavie virus” is a subset of envelope viruses. They are generally viruses found in animals transmitted to human through an insect that have infected humans by acquiring a lipid bilayer envelope. Examples of flavie viruses include Yellow Fever, Dengue, West Nile, and encephalitis viruses.

As used herein, the term “non-envelope virus” refers to a virus lacking a lipid bilayer. In non-envelope viruses, the capsid mediates attachment to and penetration into host cells. Examples of non-envelope viruses include Norwalk virus, hepatitis B, polio, and rhinoviruses.

A “patient,” “subject” or “host” to be treated by the subject method may mean either a human or non-human animal.

As used herein, the term “protozoan” or “protozoa” refers to a class of Protists that are defined as single-celled eukaryotic organisms that feed heterotrophically and exhibit diverse motility mechanisms. Protists exhibit an enormous range of body form, even though they are largely microscopic, mainly ranging in size from 10-200 μm and account or over 60,000 species.

As used herein, the term “bacteria” refers to a prokaryotic class of unicellular (single or chains) organisms or microbes that lack organelles and fall into two general classes Gram-positive and Gram negative based on the chemically staining properties of their cell wall.

As used herein, the term “pathogen” refers to a microbial organisms that are capable of infecting and residing in specific hosts and causing disease or dysfunction of the host system.

As used herein, the term “prion” refers to a proteinaceous infectious particles that are malformed proteins that form plaques or amyloids on cerebral neuronal tissues leading to disruption of neuron function and apoptosis. They are the cause of a number transmissible of neurodegenerative diseases in mammals, such as bovine spongiform encephalopathies (BSE).

The term “preventing”, when used in relation to a condition, such as cancer, an infectious disease, or other medical disease or condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The term “synergistic” is art recognized and refers to two or more components working together so that the total effect is greater than the sum of the components.

The term “treating” is art-recognized and refers to curing as well as ameliorating at least one symptom of any condition or disorder

The term “virus” is art recognized and refers to non-cellular biological entities lacking metabolic machinery of their own and reproduce by using that of a host cell. Viruses comprise a molecule of nucleic acid (DNA or RNA) and can be envelope or non-envelope viruses.

A “patient,” “subject” or “host” to be treated by the subject method includes either a human or non-human animal.

The compounds of the present invention may be used in the form of pharmaceutically-acceptable salts derived from inorganic or organic acids. The term “pharmaceutically-acceptable salt” includes those salts that are, within the scope of sound medical judgment, suitable for use in contact with the tissues of humans and lower animals without undue toxicity, irritation, and allergic response, and are commensurate with a reasonable benefit/risk ratio. Pharmaceutically-acceptable salts are well known in the art. For example, S. M. Berge, et al. describe pharmaceutically-acceptable salts in J Pharm Sci, 1977, 66:1-19. The salts may be prepared in situ during the final isolation and purification of the compounds of the invention or separately by reacting a free base function with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethanesulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates, such as dimethyl, diethyl, dibutyl and diamyl sulfates; long-chain halides such as decyl, lauryl, myristyl and stearyl chlorides, bromides and iodides; or arylalkyl halides, such as benzyl and phenethyl bromides and others. Water- or oil-soluble or -dispersible products are thereby obtained.

Examples of acids that may be employed to form pharmaceutically acceptable acid addition salts include such inorganic acids as hydrochloric acid, hydrobromic acid, sulfuric acid and phosphoric acid and such organic acids as oxalic acid, maleic acid, succinic acid, and citric acid.

The present invention includes all salts and all crystalline forms of such salts. Basic addition salts can be prepared in situ during the final isolation and purification of compounds of this invention by combining a carboxylic acid-containing group with a suitable base such as the hydroxide, carbonate, or bicarbonate of a pharmaceutically acceptable metal cation or with ammonia or an organic primary, secondary, or tertiary amine. Pharmaceutically acceptable basic addition salts include cations based on alkali metals or alkaline earth metals such as lithium, sodium, potassium, calcium, magnesium, and aluminum salts, and nontoxic quaternary ammonia and amine cations including ammonium, tetramethylammonium, tetraethylammonium, methylamine, dimethylamine, trimethylamine, triethylamine, diethylamine, and ethylamine. Other representative organic amines useful for the formation of base addition salts include ethylenediamine, ethanolamine, diethanolamine, piperidine, and piperazine.

### Compounds

Isolated compounds have been identified from extracts showing antiviral activity. Compounds of the present invention have also been synthesized (>98% purity) and show anti-influenza activity. Compounds of the present invention include flavononols, such as Tristenonol.

The pure and isolated flavononol compounds of the present invention are represented by formula I:

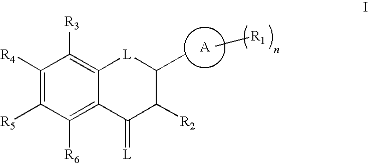

wherein, independently for each occurrence:

R₁ represents alkoxy, alkenyloxy, alkynyloxy, aryloxy, arylalkyloxy, hydroxy, —OC(O)—R₇, alkyl, alkenyl, alkynyl, acetyl, formyl, halide, cyano, nitro, SH, amino, amido, sulfonyl, or sulfonamido;

R₂ represents OH or

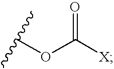

R₃, R₄, R₅, and R₆ represent H, hydroxy, alkoxy, alkenyloxy, alkynyloxy, aryloxy, aralkyloxy; —OC(O)—R₇, alkyl, alkenyl, alkynyl, aralkyl, acetyl, formyl, halide, cyano, nitro, SH, amino, amido, sulfonyl, or sulfonamido;

R₇ represents H, alkyl, alkenyl, alkynyl, cycloalkyl, aryl, arylalkyl or a carbohydrate;

A represents an aryl group;

L represents O, S, or NR;

R represents H, hydroxy, alkyl, alkenyl, alkynyl, aralkyl, acetyl, formyl, or sulfonyl;

X represents a carbohydrate, a cycloalkyl, or a cycloalkenyl group; and

n represents an integer from 1 to 5, inclusive;

wherein any of the aforementioned alkoxy, alkenyloxy, alkynyloxy, aryloxy, aralkyloxy, alkyl, alkenyl, alkynyl, cycloalkyl, cycloalkenyl, aryl and aralkyl groups may be optionally substituted with one or more groups selected from the group consisting of hydroxy, alkoxy, alkenyloxy, alkynyloxy, aryloxy, aralkyloxy; halide, formyl, acetyl, cyano, nitro, SH, amino, amido, sulfonyl, or sulfonamido.

In another embodiment, the esterified flavonolol compounds of the present invention are represented by formula I, wherein, independently for each occurrence:

R₁ represents H, alkoxy, aryloxy, aralkyloxy, hydroxy, —OC(O)—R₇, alkyl, acetyl, formyl, or halide;

R₂ represents

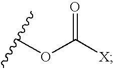

R₃, R₄, R₅, and R₆ represent H, alkoxy, aryloxy, aralkyloxy; —OC(O)—R₇, alkyl, aralkyl, acetyl, formyl, or halide;

R₇ represents H, alkyl, aryl, or arylalkyl;

A represents an aryl group;

L represents O;

X represents a carbohydrate, a cycloalkyl, or a cycloalkenyl group; and

n represents an integer from 1 to 5, inclusive;

wherein any of the aforementioned alkoxy, alkenyloxy, alkynyloxy, aryloxy, aralkyloxy, alkyl, alkenyl, alkynyl, aryl, aralkyl, cycloalkyl and cycloalkenyl groups may be optionally substituted with one or more groups selected from the group consisting of hydroxy, alkoxy, alkenyloxy, alkynyloxy, aryloxy, aralkyloxy; halide, formyl, acetyl, cyano, nitro, SH, amino, amido, sulfonyl, or sulfonamido.

The carbohydrate may be a monosaccharide such as arabinose, lyxose, ribose, rhamnose, deoxyribose, xylose, ribulose, xylulose, allose, altrose, galactose, glucose, gulose, idose, mannose, talose, fructose, mannose, psicose, sorbose, or tagatose. In another embodiment, the carbohydrate may be a disaccharide such as sucrose, lactose, maltose, trehalose or cellobiose. In another embodiment, the carbohydrate may be an oligosaccharide such as raffinose, maltodextrin, and cyclodextrin. In another embodiment, the carbohydrate may be a polysaccharide such as starch, glycogen, dextran, and cellulose.

In another embodiment, R₂ is OH.

In another embodiment, the flavononol compounds are represented by formula I, wherein L is O.

In another embodiment, the flavononol compounds are represented by formula I, wherein R₃, R₄, R₅ and R₆ are each independently H or hydroxy, wherein at least two of R₃, R₄, R₅ and R₆ are hydroxy.

In another embodiment, the flavononol compounds are represented by formula I, wherein R₁ is hydroxy, and n is equal to 2 or 3.

In another embodiment, the flavononol compounds are represented by formula I, wherein A is a benzene ring.

In another embodiment, the flavononol compounds are represented by formula I, wherein X is a carbohydrate.

In another embodiment, the flavononol compounds are represented by formula I, wherein X is a cycloalkyl or cycloalkenyl group; and wherein the cycloalkyl or cycloalkenyl group is substituted with 1 to 3 hydroxy groups.

In another embodiment, the flavononol compounds of the present invention are represented by formula Ia:

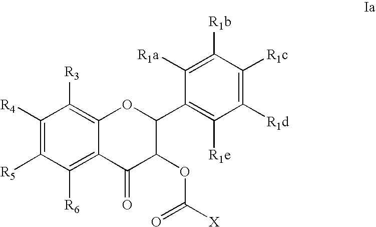

wherein, independently for each occurrence:

R₁ₐ, R_{1b}, R_{1c}, R_{1d}, R₁ₑ represent H, hydroxy, alkoxy, aralkyloxy, or aryloxy;

R₃, R₄, R₅, and R₆ represent H, hydroxy, alkoxy, aryloxy, or aralkyloxy; and

X represents a carbohydrate, a cycloalkyl, or a cycloalkenyl group;

wherein any of the aforementioned alkoxy, aryloxy, aralkyloxy may be optionally substituted with one or more groups selected from the group consisting of hydroxy, alkoxy, aryloxy, aralkyloxy; halide, formyl, acetyl, cyano, nitro, SH, amino, amido, sulfonyl, or sulfonamido. In another embodiment, the flavononols of the present invention are represented by formula Ia, wherein independently for each occurrence:

R₁ₐ, R_{1b}, R_{1c}, R_{1d}, and R₁ₑ represent H, hydroxy, alkoxy, aralkyloxy, or aryloxy; provided that at least two of R₁ₐ, R_{1b}, R_{1c}, R_{1d}, and R₁ₑ are hydroxy;

R₃, R₄, R₅, and R₆ represent H, hydroxy, alkoxy, aryloxy, or aralkyloxy, provided that at least two of R₃, R₄, R₅, and R₆ are hydroxy; and

X is carbohydrate, cycloalkyl, or cycloalkenyl;

wherein any of the aforementioned alkoxy, aryloxy, aralkyloxy, cycloalkyl, or cycloalkenyl may be optionally substituted with one or more groups selected from the group consisting of hydroxy, alkoxy, aryloxy, aralkyloxy; halide, formyl, acetyl, cyano, nitro, SH, amino, amido, sulfonyl, or sulfonamido.

In another embodiment, the flavononol compounds of the present invention are represented by formula Ia, wherein: R₁ₐ, R_{1b}, R_{1c}, R_{1d}, and R₁ₑ represent H or hydroxy, and three of R₁ₐ, R_{1b}, R_{1c}, R_{1d}, and R₁ₑ are hydroxy.
In another embodiment, the flavononol compounds of the present invention are represented by formula Ia, wherein: R₃, R₄, R₅, and R₆ represent H or hydroxy, and two of R₃, R₄, R₅, and R₆ are hydroxy.
In another embodiment, the flavononol compounds of the present invention are represented by formula Ia, wherein: X is a carbohydrate selected from the group consisting of a monosaccharide, a disaccharide, an oligosaccharide, and a polysaccharide.

In another embodiment, X is a carbohydrate selected from the group consisting of arabinose, lyxose, ribose, rhamnose, deoxyribose, xylose, ribulose, xylulose, allose, altrose, galactose, glucose, gulose, idose, mannose, talose, fructose, psicose, sorbose, tagatose, sucrose, lactose, maltose, trehalose or cellobiose, raffinose, maltodextrin, cyclodextrin, starch, glycogen, dextran, and cellulose.

In yet another embodiment, X is rhamnose.

In another embodiment, X is a cycloalkyl or cyloalkynyl group, wherein the cycloalkyl or cycloalkenyl group may be substituted with one to three hydroxy groups.

In another embodiment, the flavononol compounds of the present invention are represented by formula Ib,

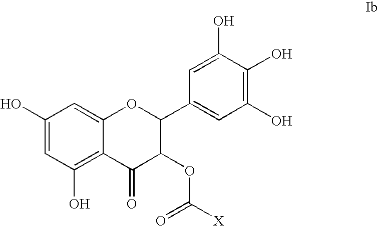

wherein X represents a carbohydrate, a cycloalkyl, or a cycloalkenyl, wherein the cycloalkyl or cycloalkenyl may be substituted with one to three hydroxy groups. The carbohydrate may be a monosaccharide such as arabinose, lyxose, ribose, rhamnose, deoxyribose, xylose, ribulose, xylulose, allose, altrose, galactose, glucose, gulose, idose, mannose, talose, fructose, psicose, sorbose, or tagatose. In another embodiment, the carbohydrate may be a disaccharide such as sucrose, lactose, maltose, trehalose or cellobiose. In another embodiment, the carbohydrate may be an oligosaccharide such as raffinose, maltodextrin, and cyclodextrin. In another embodiment, the carbohydrate may be a polysaccharide such as starch, glycogen, dextran, and cellulose.

Esterification on the 3′-O of Ring C on the flavononol proceeds through reaction of the acid form of the above listed carbohydrate under standard esterification conditions.

In another embodiment, X is a cyclohexyl or cyclohexenyl. In another embodiment, X is:

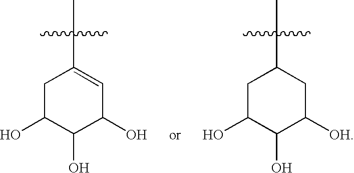

In a further embodiment, the flavononol of the present invention is:

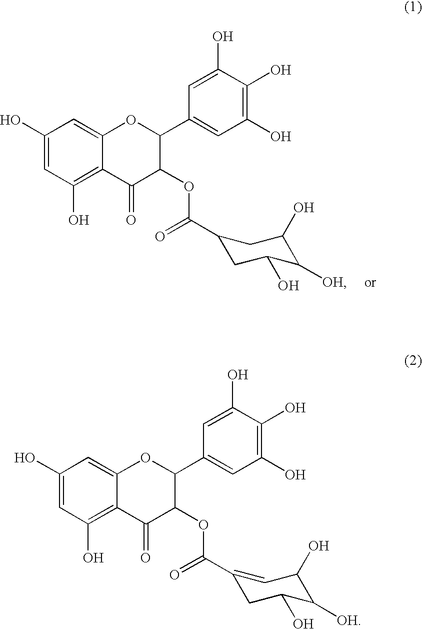

The aforementioned compounds may be pure and isolated, e.g., by chemical synthesis and/or extraction from a botanical, or the compounds may be present in a mixture. In some embodiments, the aforementioned compounds are present in an amount of about 5 to 90% in a mixture, such as a mixture obtained by extraction of a botanical. In other embodiments, the aforementioned compounds may be present in an amount of about 5, 10, 15, 20, 25, 30, 35, 40, 45, 50, 55, 60, 65, 70, 75, 80, 85, 90 or 95% in a mixture.

In another embodiment, the compound is:

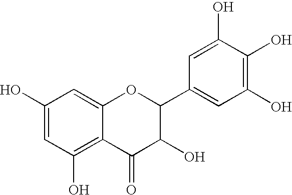

Isolation of Compounds from Botanicals

The flavononols and leucoanthocyanidins can be obtained by extraction and purification from a botanical, such as elderberry, to obtain, for example, the 479.5 m/z [M+H] flavononols and leucoanthocyanidins. A botanical extract (powder, paste or liquid) is extracted with warm water (40° C.) and the eluate is loaded onto Celite, and the pellet is discarded. The Celite-bound material is washed with low ionic strength Tris-HCl buffer (pH 8.2), and the washed material discarded. The Celite-bound fraction is released with high ionic strength K-phosphate buffer, collected and then loaded onto hydroxyapatite. The flavononol or leucoanthocyanidin is collected with an increasing gradient of K-phosphate buffer, and the lower molecular weight (<250 MW) phenolic fraction is discarded.

The flavononols and leucoanthocyanidins can also be obtained from an LH20 resin purification of the polyphenolic fraction from a botanical or other source. LH-20 should be conditioned with ethanol, and a gradient of water and organic solvent (methanol, ethanol, or acetonitrile) can be used for elution of compounds of the present invention from the LH-20 resin.

### Synthesis of Compounds of the Present Invention

The compounds obtained from an extract may be further purified and/or modified by synthetic organic methods well-known in the art.

The compounds of the invention may also be obtained by synthetic organic method well-known in the art. For example, Scheme I depicts a general route to the synthesis of flavononols. The starting material is an Rb-substituted acetyl phenone (i) and benzaldehyde, where Rb-groups are alkoxy, alkenyloxy, alkynyloxy, aryloxy, arylalkyloxy, hydroxy, —OC(O)—R₇, alkyl, alkenyl, alkynyl, acetyl, formyl, halide, cyano, nitro, SH, amino, amido, sulfonyl, or sulfonamido. The Rb-groups may additionally be one the aforementioned groups protected with a suitable protecting group to prevent undesired side reactions. For example, OH may be protected by protecting groups such as methoxymethyl (MOM), or NH₂ may be protected with CBZ, etc. The starting material (i) undergoes a base-catalyzed aldol condensation or acid-mediated adolization with the substituted benzaldehyde to yield a chalcone (ii). (See March 1994, Streitweiser 1992). The chalcone is then expoxidized to form epoxy chalcone (iii) or subjected to based-catalyzed cyclization to form flavonone (iv). (See March 1994, Carey and Sundberg 1992). The epoxy chalcone is subjected to either acid, free radical or Lewis acid-catalyzed cyclization to yield flavononol (v). (See March 1994, Carey and Sundberg 1992). Flavonone (iv) undergoes an oxidation reaction to yield the flavononol. (See March 1994, Carey and Sundberg 1992).

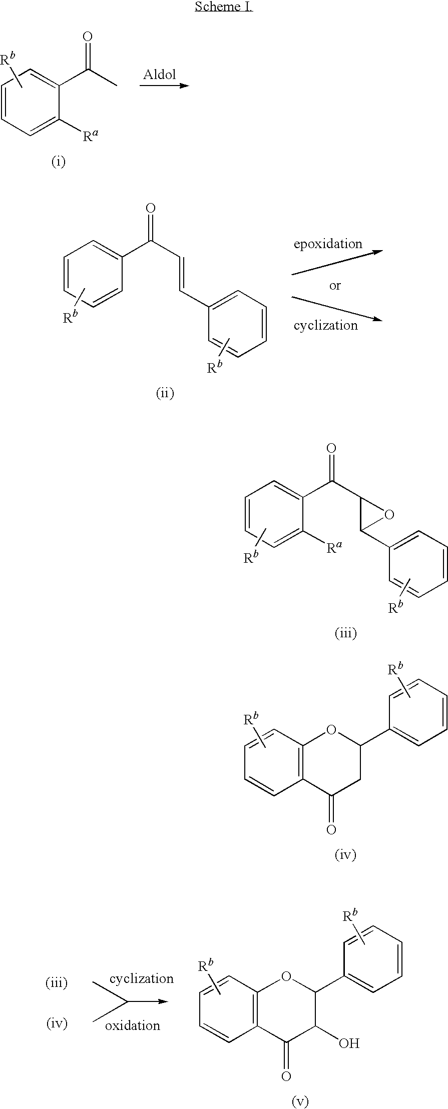

The flavononol (v) as described in Scheme I is esterified under acid catalysis with a carboxylic acid, for example, 3,4,5-trihydroxy cyclohexane carboxylic acid (e.g. shikimic acid) or glycosylated on the 3-OH group of the C ring to yield esterified flavononol (vii). (See March 1994, Streitweiser 1992). Additionally, the flavononol can be reduced at the C-2 carbonyl to yield a leucoanthocyanidin (vii). (See March 1994, Carey and Sundberg 1992). The flavononol and leucoanthocyanidin compounds can be further separated and purified so as to obtain pure and isolated anthocyanadins by methods known in the art, such as flash column chromatography, HPLC, recrystallization, etc.

Scheme II represents a synthetic method used to obtain a specific flavononol, the Tristenonol aglycone. The Tristenonol aglycone was synthesized in five steps by coupling methoxymethyl (MOM) protected acetophenone and benzaldehyde, 10 and 12 respectively. The chalcone formed through this reaction was epoxidized using hydrogen peroxide to give compound 14, and the compound 14 was cyclized with the aryl OH (from MOM deprotection during the same reaction) to give the Tristenonol aglycone (15) in 66% overall yield.

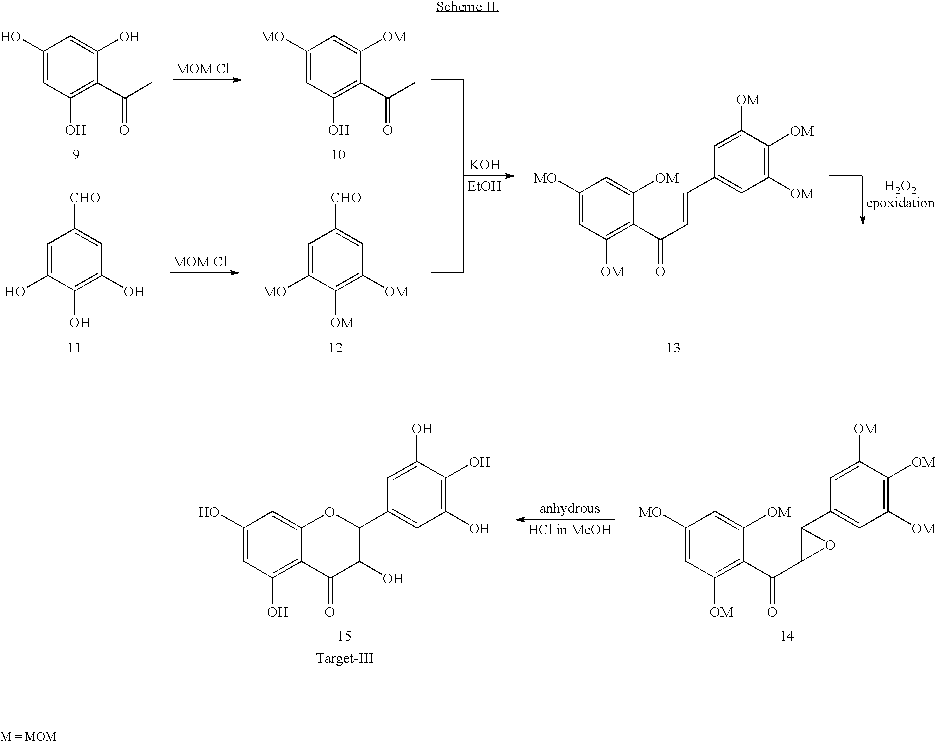

The esterified flavononols of the present invention may be prepared from flavononol (v) of Scheme I according to Scheme III:

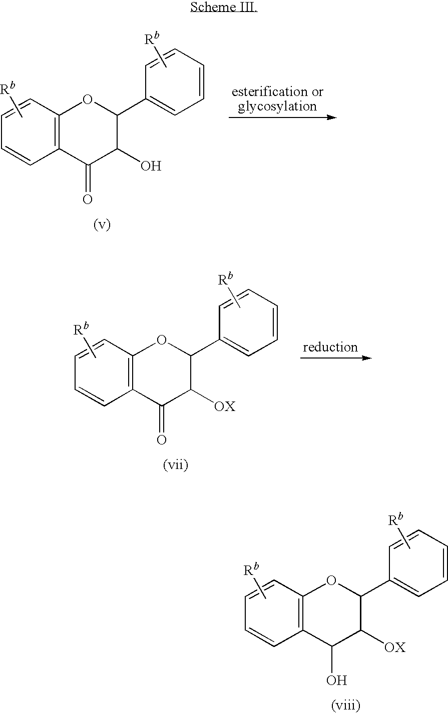

### Inhibition of Human Influenza A (H1N1) Virus Infection

A focus-forming assay was used to characterize the anti-influenza virus activity of the aforementioned compounds. Human influenza A virus subtype /PR/8/34 H1N1 were pre-incubated for 1 hour with two-fold serial dilutions of extract prior to delivery to target MDCK cell cultures. Virus infection was visualized in MDCK target cells using an antibody coupled calorimetric reaction. All extracts were buffered to pH 7.0-7.2 with HEPES buffer (pH 7.2) prior to use in focus-forming assays to ensure that viral inhibitory effects were not due to a pH-triggered inactivating conformational change in the virus. The buffer conditions did not inhibit virus entry in control experiments. Infectious events were scored over a concentration range of compounds to generate viral infection inhibition curves, and IC₅₀ and IC₁₀₀ values for the different compounds. The extract containing compounds of the present invention inhibited H1N1 viral infection as a 50% inhibitory concentration (IC50) and IC100 of 270±35 μg/ml (±1 SD) and 1262±81 μg/ml (±1 SD), respectively. Importantly, 100% of viral infection was inhibited. The synthetic Tristenonol aglycone was also subjected to focus-forming inhibition and plaque reduction assays against the H1N1 virus. The Tristenonol aglycone achieved 50% inhibition of H1N1 infection at a concentration of 2.8 μg mL⁻¹ (5.4 μM) (Table 1). Tristenonol had an IC₅₀ value 30×higher (less active) than Oseltamivir and around 3×lower than Amantadine (Table 1), which are two commonly used anti-influenza medications.

TABLE 1
Inhibition values (IC₅₀) of influenza A by Tristenonol, Oseltamivir,
and Amantadine using a foci-reduction assay.

| Compound Name | IC₅₀ μg/ml) | IC₅₀ (μM) |
|---|---|---|
| Botanical extract containing | 270 | NA |
| compounds of the present invention | | |
| Compound of the present invention | 2.8 | 5.4 |
| (Tristenonol aglycone) | | |
| Oseltamivir | 0.1 | 0.32 |
| Amantadine | 4.7 | 27 |

NA = not applicable.

### Inhibition of Avian Influenza A (H5N1) Virus Infection In Vitro

The focus-forming assay was used also to characterize the activity of the aforementioned compounds of the present invention against avian flu. Avian influenza A virus reassortant Indo/05/2005 (H5N1)/P8-IBCDC-RG2 reference strain was treated as described for the H1N1 viruses. A dose-dependent inhibition of H5N1 infection was obtained with the botanical extract with an IC₅₀ value of 475±20 μg/ml (±1 SD), and an IC₁₀₀ value of 1,200±75 μg/ml (±1 SD).

To verify that the viral inhibitory effects were not due to cellular toxicity due to the extractor the pure compounds of the present invention the materials was tested using a standard MTT calorimetric cell viability assay. No statistically significant cellular toxicity was observed over the concentration range that inhibited virus infection in vitro.

### Direct Binding of Compounds to H1N1

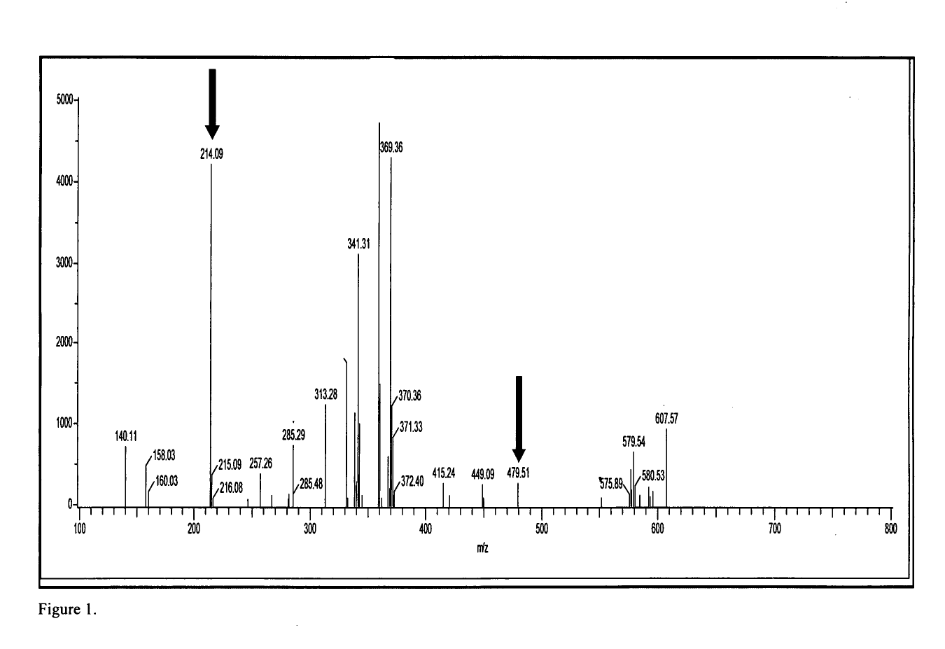

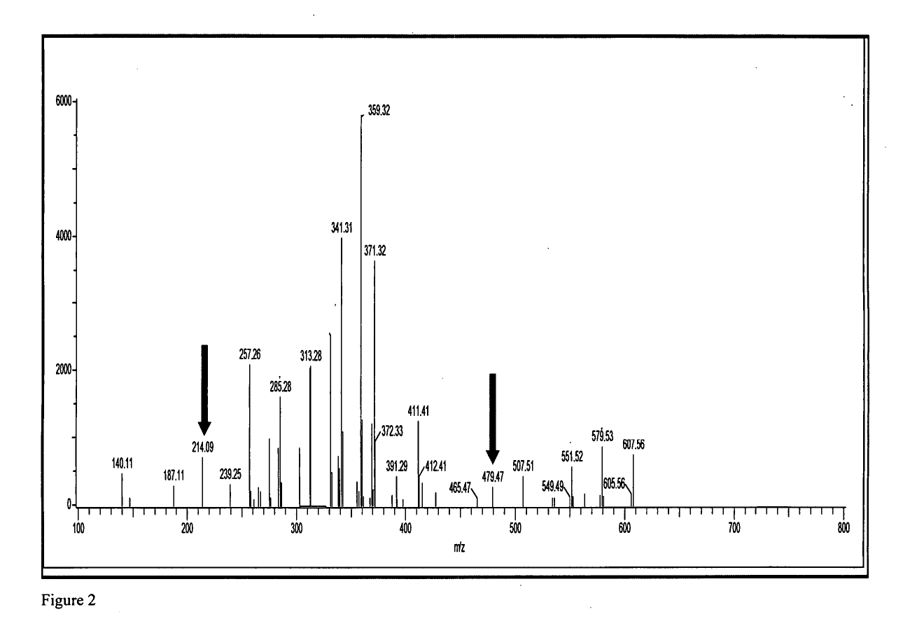

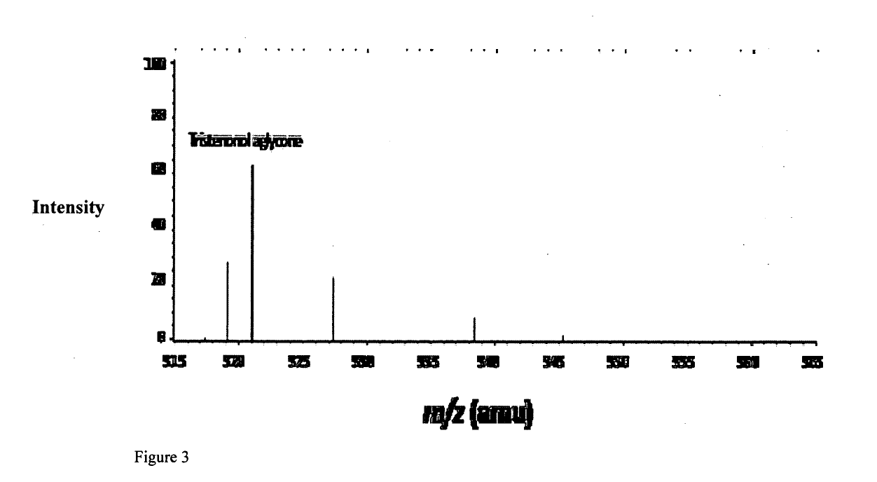

Through the use of the Direct Binding Assay and DART TOF mass spectrometry, it was possible to determine which compounds from the botanical extracts were binding to the H1N1 and H5N1 viral particles (FIGS. 1 and 2, respectively). Compounds from the present invention, present in botanical extracts, that bind to the H1N1 and H5N1 viral particles include certain flavononols (FIGS. 1 and 2, respectively). The nature and chemical characterization of the bound compounds is provided below. In a similar manner we examined the direct binding of the pure compounds of the present invention (e.g., the aglycone of the flavononol) to H1N1 virions. In FIG. 3, a DART TOF mass spectrum fingerprint of H1N1 virions incubated in 100 μg/ml of the aglycone of Tristenonol shows that the Tristenonol aglycone does bind directly to the virus surface. In a similar manner we examined the Avian flu H5N1 virus using the direct binding assay to determine the compounds that bind to the H5N1 virus (FIG. 2). As with H1N1, the dominant compounds that bind to the H5N1 particles include flavononols (FIG. 2 arrows).

### Direct Binding and Re-Infection Studies

For the direct binding assay, 100 μL of virus (3×10⁵ PFU) was incubated for approximately 1 hour at room temperature with 200 μL solution of compounds of the present invention at 100 μg/mL (IC₁₀₀). In addition, a virus no drug control was incubated in parallel. After the incubation, each virus/test compound (or media) mixture was added to a 100 kD Amicon filter column (supplied by the Sponsor) and centrifuged at 20° C., 5,000 rpm for 15 minutes. The flow through from each column was collected and saved for use as the negative control for the infections. Each column was then washed with media and centrifuged again at 20° C., 5,000 rpm for 15 minutes. The second flow through was discarded and a second wash was performed. After the second wash, the volume remaining in the upper column chamber was collected and brought up to a total volume of 300 μL in media (equivalent to the starting volume). Four ten fold dilutions (starting at a 1:20) in DMEM-0 were prepared for each sample. The negative control samples were diluted 1:20 in DMEM-0. In addition, a virus control sample (unfiltered) was prepared at 200 pfu/mL. All samples were inoculated onto 12-well plates as described above (section E) and immunostained as described for the foci inhibition assay (section G). Sample titers were calculated by dividing the total number of plaques from all counted wells by the theoretical volume of the test sample represented by the counted wells, and are reported as FFU/mL. The FFU/mL were reduced 80% relative to controls when H1N1 was incubated in a 100 μg/mL solution of compounds of the present invention, and washed free of components that do not bind to the influenza virus surface

### Structural Characterization of Compounds Bound to H1N1 and H5N1

The washed H1N1 and H5N1 virions that had been incubated in the presence of the aforementioned compounds and other compounds revealed the presence of several bound flavonoids (285.2, 303.3, 313.3, 331.3, 341.3, and 359.3 m/z [M+H]) and three flavonoid dimers (m/z [M+H], 551.4, 579.4, 607.4) or proanthocyanidins, and an esterified flavononol or leucoanthocyanidin (m/z [M+H], 479.4) (FIGS. 1 and 2, respectively). There is no detectable difference in the classes of compounds that bind to H1N1 and H5N1 based on the DART analyses (FIGS. 1 and 2). DART AccuTOF-MS MassCenterMain software was used to determine the molecular formulas of the compounds bound to H1N1 and H5N1 virions, while ESI-Linear Ion MS was used for confirmation of these compounds. In addition, DART TOF-MS and ESI-Linear Ion MS were conducted on a proanthocyanidin B₂ standard (Chromadex, Inc.). It was found that an esterified flavononol or a leucoanthocyanidin was among the novel compounds that bind to both H1N1 and H5N1 viral surfaces (FIGS. 1 and 2). The chemical structures of the identified esterified flavononols that bind to H1N1 and H5N1 were determined based upon isotope matching of the determined molecular formulas from the DART AccuTOF-MS.

### Structure of a Synthetic Aglycone of Flavononol, Tristenononol

The aglycone of one of the flavononols was synthesized to >98% purity, confirmed by HPLC. The structure of this flavononol was identical to the aglycone of a flavononol and the structure was confirmed by proton and carbon NMR. The proton NMR confirms the presence of four aromatic protons, as well as one sp3 hybridized proton neighboring an oxygen and adjacent to a ketone, and one sp3 hybridized proton neighboring an oxygen and an aromatic ring. The carbon NMR confirms the presence of twelve aromatic carbons, one ketone, and two carbons bonded to oxygen atoms. Collectively, the NMR data shows the proper coupling for the appropriate substitution patterns on the aromatic rings as well as the proper number and types of carbon atoms for the aglycone of Tristenonol.

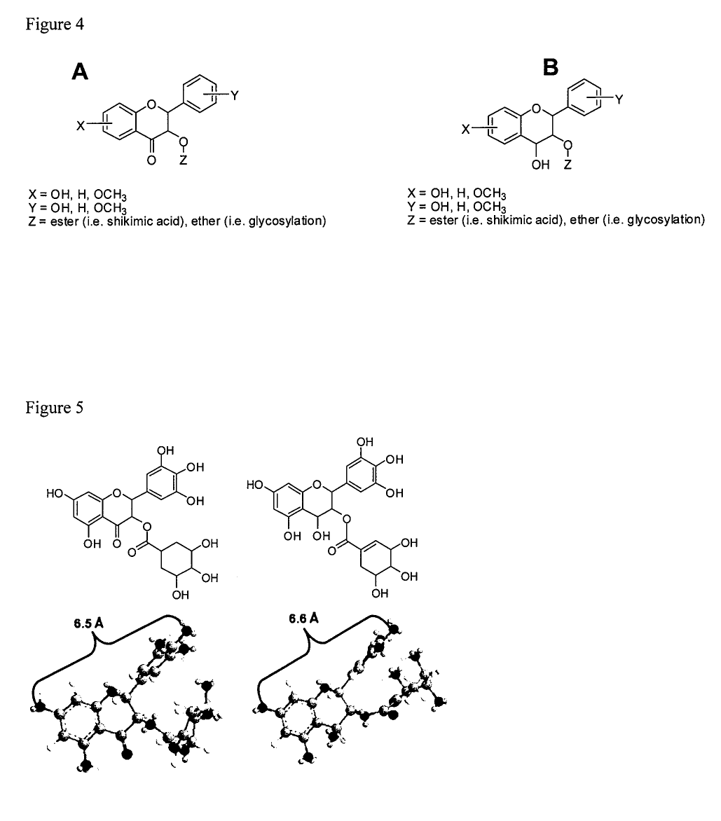

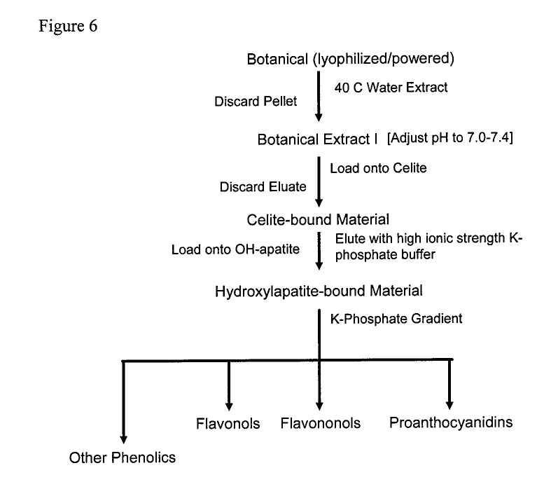

Compounds of the present invention can also be esterified or glycosylated, likely at the 3′-position of the central flavonoid C-ring as seen in FIG. 4. The flavononol is most likely esterified with shikimic acid or glycosylated with rhamnose. This flavononol ester or glycoside is uniquely bound by influenza viruses, and is determined to not bind to the surface of other enveloped or non-enveloped viruses investigated to date.

The 2-D structure of the flavononol (m/z H+, 479.4) is compared with its free energy 3-D structure in FIG. 5. The 3-D structure reveals that the phenol rings form an axis with the distance between the phenol rings (FIG. 5) of 10 Å. Based on previous work (Roschek, W., Li, D., and Alberte R S. 2008. Phytochemistry, in review; Alberte, R S. and Smith, R D 2006 Anti- adhesion and Proadhesion Combinatorial Compounds. U.S. Pat. No. 7,132,567, this is most likely the binding domain of this molecule, and as such would leave the shikimic acid domain free. This free energy structure would also meet the binding domain requirements for known pathogen adhesins (e.g., Stephens, J, Cooper, A. L., Basler, C., Taubenberger, J. K., Palese, P. and Wilson, A. Science 303:1866-1870 2004 for HINI), and would be consistent the structures of known classes of bisphenol anti-adhesin compounds (Roschek, W., Li, D. and Alberte R S. 2008. Phytochemistry, in review; Alberte, R S. and Smith, R. D. 2006. Anti-adhesion and Proadhesion Combinatorial Compounds. U.S. Pat. No. 7,132,567) that show high anti-adhesion/anti-infection activity against a range of enveloped viruses including influenza. All of these compounds with high anti-infective activity (IC₅₀ values in the low micromolar or high nanomolar range) possess inter-phenolic ring distances between 8 and 16 Å.

As discussed previously, DART TOF-MS was used to characterize the compounds of the present invention to determine their specificity for influenza viruses. Examinations of four non-influenza viruses, Dengue, Herpes and HIV-1, and Rhinovirus revealed that the flavononols or leucoanthocyanidins of the present invention do not bind, therefore supporting that these chemistries bind uniquely to influenza viruses. It is hypothesized that the compounds of the present invention are binding to influenza hemaglutinin proteins on the surface of the virion particles. Hydrophobic binding pockets have been described that would readily accommodate the flavononols and leucoanthocyanidins of the present invention. Minimum free energy analysis revealed that the aglycone of Tristenonol forms an axis with an inter-phenolic ring distance of 10.9 Å, respectively (FIG. 5). This distance is well within the size constraints of the hemaglutinin (HA) binding domain pocket (14-15 Å) of influenza viruses (J. Stevens, A. L. Corper, C. F. Basler, et al., 2004. Structure of the Uncleaved Human H1 Hemaglutinin from the Extinct 1918 Influenza Virus. Science. 303:1866-1870), which is responsible for host cell receptor binding and viral entry. The phenolic regions of Tristenonol therefore, most likely binds to the viral mannose-rich HA binding domains and as such, this proposed bound orientation of Tristenonol would leave the esterified or glycosylated functionality of Tristenonol free to interact with immune receptors, potentially increasing an immune response to the viral particles in vivo (D. J. Vigerust and V. L. Shepherd, 2007. Virus glycosylation: role in virulence and immune interactions. Trends in Microbiology. 15:211-218; H. Kolodziej and A. F. Kiderlen, 2005. Antileishmanial activity and immune modulatory effects of tannins and related compounds on Leishmania parasitised RAW 264.7 cells. Phytochemistry. 66:2056-2071; A. A. E. Bertelli, C. Mannari, S. Santi, et al., 2008. Immunomodulatory activity of Shikimic acid and Quercitin in comparison with Oseltamivir (Tamiflu) in an “in vitro” model. Journal of Medical Virology. 80:741-745).

Collectively the evidence indicates that these novel compounds serve as anti-adhesins that are targeted to the influenza virus particle domains involved in host cell receptor recognition and binding, and offer a new therapeutic target for drug development. Anti-adhesin compounds have been described for Gram-positive and Gram negative bacteria and fungal spores, and these previously described compounds function by binding to the bacteria masking their ability to adhere to manmade surfaces or to infect cells.

### Pharmaceutical and Personal Healthcare Formulations

The antiinfective compositions of the present invention may be administered by various means, depending on their intended use, as is well known in the art. For example, if compositions of the present invention are to be administered orally, they may be formulated as pharmaceutical compositions, such as tablets, capsules, granules, powders or syrups. Alternatively, formulations of the present invention may be administered parenterally as injections (intravenous, intramuscular or subcutaneous), drop infusion preparations or suppositories. For application by the ophthalmic mucous membrane route, compositions of the present invention may be formulated as eye drops or eye ointments. These formulations may be prepared by conventional means, and, if desired, the compositions may be mixed with any conventional additive, such as an excipient, a binder, a disintegrating agent, a lubricant, a corrigent, a solubilizing agent, a suspension aid, an emulsifying agent or a coating agent.

In formulations of the subject invention, wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

Subject compositions may be suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of composition that may be combined with a carrier material to produce a single dose vary depending upon the subject being treated, and the particular mode of administration.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations which are suitable for vaginal administration also include pessaries, tampons, creams, gels, pastes, foams or spray formulations containing such carriers as are known in the art to be appropriate.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants that may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions of the present invention may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions.

Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this invention suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

The dosage of any compositions of the present invention will vary depending on the symptoms, age and body weight of the patient, the nature and severity of the disorder to be treated or prevented, the route of administration, and the form of the subject composition. Any of the subject formulations may be administered in a single dose or in divided doses. Dosages for the compositions of the present invention may be readily determined by techniques known to those of skill in the art or as taught herein.

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 10 mg per kg.

An effective dose or amount, and any possible affects on the timing of administration of the formulation, may need to be identified for any particular composition of the present invention. This may be accomplished by routine experiment as described herein, using one or more groups of animals (preferably at least 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including composition, amounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The use of the subject compositions may reduce the required dosage for any individual agent contained in the compositions because the onset and duration of effect of the different agents may be complimentary.

Toxicity and therapeutic efficacy of subject compositions may be determined by standard pharmaceutical procedures in cell cultures or experimental animals, e.g., for determining the LD₅₀ and the ED₅₀.

The data obtained from the cell culture assays and animal studies may be used in formulating a range of dosage for use in humans. The dosage of any subject composition lies preferably within a range of circulating concentrations that include the ED₅₀ with little or no toxicity. The dosage may vary within this range depending upon the dosage form employed and the route of administration utilized. For compositions of the present invention, the therapeutically effective dose may be estimated initially from cell culture assays.

Applications include cosmetics and other over-the-counter products for human and animal application. Preservatives are used to prevent the growth of bacteria and fungi that may result in product contamination and deterioration. Compounds of the present invention can be used in combination with an existing preservative such as: alcohols; benzoic acid; chlorhexidine; diazolidinyl urea; dimethylol dimethylhydantoin-1,3-bis; isothiazolones; mercurials; parabens; phenolic compounds; quaternary ammonium compounds; and triclosan. Treatment concentrations could be reduced when these agents are used in combination with compounds of the present invention.

### Methods of Treatment

The present invention also relates in part to a method of treating an infection in a subject comprising administering to a subject in need thereof a therapeutically effective amount of a compound or composition of the present invention.

In a further embodiment, the infection is a viral infection caused by an envelope virus, while in other embodiments, the viral infection caused by a non-envelope virus. In a further embodiment, the infection is a viral infection caused by an envelope virus selected from the group consisting of human influenza, avian influenza, HIV, SARs, HPV, herpes simplex virus (HSV-1) and related Herpes viruses (HSV-2, EBV, CMV, HHV-6, HHV-8), Herpes zoster, Hepatitis A and C, Dengue (1-4), Yellow Fever, West Nile, and other encephalitis viruses. In a further embodiment, the infection is a viral infection caused by a non-envelope virus selected from the group consisting of Norwalk virus, polio, adenoviruses, and rhinoviruses.

In a further embodiment, the infection is a bacterial infection caused by bacteria that include a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Bordetella, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrhea, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella spp., Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, B. pertussis, B. parapertussis, B. bronchiseptica Burkholderia cepacia, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii, Helicobacter pylori or Chlamydia trachomitis.

Non-limiting examples of illnesses caused by a microbial illness include otitis media, conjunctivitis, pneumonia, bacteremia, meningitis, sinusitis, pleural empyema and endocarditis, as well as meningitis, such as for example infection of cerebrospinal fluid. Also treatable are biofilm based infections as well as non-biofilm applications (e.g. bacterial meningitis, where antibiotics kill the bacteria, but the dead/lysed bacteria induce a very strong inflammatory response because the adhesins still bind to cell receptors causing brain swelling; compositions of the present invention would improve the therapeutic benefit and reduce risks even though no biofilm intervention mode is involved). It has been shown that lysed and/or heat killed bacteria still adhere (and induce inflammatory response) to cell receptors. Compounds of the present invention are capable of preventing such adhesion and prevent biofilm formation. Thus, by interfering with the inflammatory cascade, compositions of the present invention are useful for the treatment of such diseases as cystic fibrosis, meningitis, and oral disease. They are also useful for industrial applications where biofilm formation would lead to health related problems, such as the food industry or the water purification industry.

In a further embodiment, the infection is a fungal infection caused by B. cinerea, Penicillium sp., P. expansum, P. italicum, P. digitalum, Rhizopus sp., R. sulonifer, R. nigricans, Alternaria sp., A. alternata, A. solani, Diploidia sp., Diploidia natalenses, Monilinia sp., M. fructicola, Pseudomonas sp., P. cepacia, Xanthomonas sp., Erwinia sp. and Corynebacterium. Cladosporium sp., C. fulva, Phytophtora sp., P. infestans, Colletotricum spp., C. coccoides C. fragariae, C. gloesporioides, Fusarium spp., F. lycopersici, Verticillium spp., V. alboatrum, V. dahliae, Unicula spp., U. necator, Plasmopara spp., P. viticola, Guignardia spp., G. bidwellii, Cercospora spp., C. arachidicola, Scelrotinia spp., S. scerotiorum, Puccinia spp., P. arachidis, Aspergillus spp., A. favus, Venturia spp, V. inaequalis, Podosphaera spp., P. leucotricha, Pythiun spp., Sphaerotheca, or S. macularis.

In a further embodiment, the infection is a protozoan or related eukaryotic parasitic infection, including Entamoeba histolytica, Giardia lambila, Trichomonas vaginalis, Trypanosoma brucei T. cruzi, Leishmania donovani, Balantidium coli, Toxoplasma gondii, Plasmodium spp., Babesia microti and other water-borne protozoans, that cause certain sexually transmitted diseases, sleeping sickness (Trypanosomeniasis), Amoebiasis, Giardiasis, Trichomoniasis, African Sleeping Sickness, American Sleeping Sickness, Leishmaniasis, Balantidiasis, Toxoplasmosis, Malaria, and Babesiosis.

In a further embodiment, the infection is a prion infection selected from the group consisting of scrapie in sheep, bovine spongiform encephalopathy (BSE), transmissible mink encephalopathy (TME), chronic wasting disease (CWD) in elk and mule deer, feline spongiform encephalopathy in cats, exotic ungulate encephalopathy (EUE) in nyala, oryx, and greater kudu, Creutzfeldt-Jakob Disease (CJD), Tatrogenic Creutzfeldt-Jakob disease, Variant Creutzfeldt-Jakob disease, Familial Creutzfeldt-Jakob disease, Sporadic Creutzfeldt-Jakob disease; Gerstmann-Straussler-Scheinker syndrome (GSS), Fatal Familial Insomnia (FFI), Kuru, and Alpers syndrome.

In a further embodiment, the product is a vaccine derived from a viral ‘adhesin’ domain that is the 3-7 amino acid in length, mimicking the binding site of compounds of the present invention. In a further embodiment the binding sequences are used as antigens for vaccine production and such resulting vaccine would have broad anti-viral activity.

In a further embodiment, the subject is a vertebrate. In a further embodiment, the subject is in the class Aves. In a further embodiment, the subject is a mammal. In a further embodiment, the subject is a primate. In another aspect, the present invention relates to a method of detecting a microbial agent or amyloid with a pharmaceutical composition of the present invention. In certain embodiments, the present invention is directed to a method for formulating the pharmaceutical compositions onto a solid support in an acceptable use format for diagnosis, pathogen identification and detection. In certain other embodiments the present invention is directed to a method for formulating the pharmaceutical compositions in solution in an acceptable use format for diagnosis and pathogen detection. In another aspect, the present invention is directed to a method of making immobilized forms of the pharmaceutical compositions on non-wovens and other solid supports to achieve a disinfection and decontamination capability of air and liquid streams or systems that would include, but not be limited to filters, HVAC systems, masks, biodefense filters for personnel, buildings, water decontamination, decontamination of blood and other body fluids, and for uses in food safety.

### Additional Active Ingredients

Compositions of the present invention may further comprise additional active agents, which may work synergistically with the compounds of the present invention. Alternatively, the additional active agents may, when not provided in a composition with the inventive compounds, may be administered in conjunction with the compounds of the invention. Additional compounds include antibiotic agents that may be used in the antiinfective compositions of the present invention including cephalosporins, quinolones and fluoroquinolones, penicillins, penicillins and beta lactamase inhibitors, carbepenems, monobactams, macrolides and lincosamines, glycopeptides, rifampin, oxazolidonones, tetracyclines, aminoglycosides, streptogramins, sulfonamides, and others. Each family comprises many members.

Cephalosporins are further categorized by generation. Non-limiting examples of cephalosporins by generation include the following. Examples of cephalosporins I generation include Cefadroxil, Cefazolin, Cephalexin, Cephalothin, Cephapirin, and Cephradine. Examples of cephalosporins II generation include Cefaclor, Cefamandol, Cefonicid, Cefotetan, Cefoxitin, Cefprozil, Ceftmetazole, Cefuroxime, Cefuroxime axetil, and Loracarbef. Examples of cephalosporins III generation include Cefdinir, Ceftibuten, Cefditoren, Cefetamet, Cefpodoxime, Cefprozil, Cefuroxime (axetil), Cefuroxime (sodium), Cefoperazone, Cefixime, Cefotaxime, Cefpodoxime proxetil, Ceftazidime, Ceftizoxime, and Ceftriaxone. Examples of cephalosporins IV generation include Cefepime.

Non-limiting examples of quinolones and fluoroquinolones include Cinoxacin, Ciprofloxacin, Enoxacin, Gatifloxacin, Grepafloxacin, Levofloxacin, Lomefloxacin, Moxifloxacin, Nalidixic acid, Norfloxacin, Ofloxacin, Sparfloxacin, Trovafloxacin, Oxolinic acid, Gemifloxacin, and Perfloxacin.

Non-limiting examples of penicillins include Amoxicillin, Ampicillin, Bacampicillin, Carbenicillin Indanyl, Mezlocillin, Piperacillin, and Ticarcillin. Non-limiting examples of penicillins and beta lactamase inhibitors include Amoxicillin-Clavulanic Acid, Ampicillin-Sulbactam, Benzylpenicillin, Cloxacillin, Dicloxacillin, Methicillin, Oxacillin, Penicillin G (Benzathine, Potassium, Procaine), Penicillin V, Piperacillin+Tazobactam, Ticarcillin+Clavulanic Acid, and Nafcillin. Non-limiting examples of carbepenems include Imipenem-Cilastatin and Meropenem. A non-limiting example of a monobactam includes Aztreonam.

Non-limiting examples of macrolides and lincosamines include Azithromycin, Clarithromycin, Clindamycin, Dirithromycin, Erythromycin, Lincomycin, and Troleandomycin.

Non-limiting examples of glycopeptides include Teicoplanin and Vancomycin.

Non-limiting examples of rifampins include Rifabutin, Rifampin, and Rifapentine.

A non-limiting example of oxazolidonones includes Linezolid.

Non-limiting examples of tetracyclines include Demeclocycline, Doxycycline, Methacycline, Minocycline, Oxytetracycline, Tetracycline, and Chlortetracycline.

Non-limiting examples of aminoglycosides include Amikacin, Gentamicin, Kanamycin, Neomycin, Netilmicin, Streptomycin, Tobramycin, and Paromomycin.

A non-limiting example of streptogramins includes Quinopristin+Dalfopristin.

Non-limiting examples of sulfonamides include Mafenide, Silver Sulfadiazine, Sulfacetamide, Sulfadiazine, Sulfamethoxazole, Sulfasalazine, Sulfisoxazole, Trimethoprim-Sulfamethoxazole, and Sulfamethizole.

Non-limiting examples of other antibiotic agents include Bacitracin, Chloramphenicol, Colistemetate, Fosfomycin, Isoniazid, Methenamine, Metronidazol, Mupirocin, Nitrofurantoin, Nitrofurazone, Novobiocin, Polymyxin B, Spectinomycin, Trimethoprim, Colistin, Cycloserine, Capreomycin, Pyrazinamide, Para-aminosalicyclic acid, and Erythromycin ethylsuccinate+sulfisoxazole.

Non-limiting examples of antifungal agents that may be used in the antiinfective compositions of the present invention include antifungal agents that also act as antibiotics such as polyenes and others, and synthetic antifungal agents such as allylamines, imidazoles, thiocarbamates, triazoles, and others.

Non-limiting examples of polyenes include Amphotericin B, Candicidin, Dermostatin, Filipin, Fungichromin, Hachimycin, Hamycin, Lucensomycin, Mepartricin, Natamycin, nystatin, Pecilocin, and Perimycin.

Non-limiting examples of allylamines include Butenafine, Naftifine, and Terbinafine.

Non-limiting examples of imidazoles include Bifonazole, Butoconazole, Chlordantoin, Chlormidazole, Cloconazole, Clotrimazole, Econazole, Enilconazole, Fenticonazole, Flutirmazole, Isoconazole, ketoconazole, lanoconazole, Miconazole, Omoconazole, Oxiconazole Nitrate, Sertaconazole, Sulconazole, and Tioconazole.

Non-limiting examples of thiocarbamates include Tolciclate, Tolindate, and Tolnaftate.

Non-limiting examples of triazoles include Fluconazole, Itraconazole, Saperconazole, and Terconazole.

Non-limiting examples of other antifungal agents include Azaserine, Griseofulvin, Oligomycins, Neomycin Undecylenate, PyrroInitrin, Siccanin, Tubercidin, Viridin, Acrisorcin, Amorolfine, Biphenamine, Bromosalicylchloranilide, Buclosamide, Calcium Propionate, Chlorophenesin, Ciclopirox, Cloxyquin, Coparaffinate, Diamthazole dihydrochloride, Exalamide, Flucytosine, Halethazole, Hexetidine, loflucarban, Nifuratel, potassium iodide, propionic acid, Pyrihione, Salicylanilide, sodium propionate, Sulbentine, Tenonitrozole, Triacetin, Ujothion, undecylenic acid, and zinc propionate.

Non-limiting examples of antiviral agents that may be used in the antiinfective compositions of the present invention include Purines/Pyrimidinones and others. Non-limiting examples of Purines/Pyrimidinones include Acyclovir, Cidofovir, Cytarabine, Dideoxyadenosine, Didanosine, Edoxudine, Famciclovir, Floxuridine, Inosine Pranobex, Lamivudine, MADU, Penciclovir, Sorivudine, Stavudine, Trifluridine, Valacyclovir, Vidarabine, Zalcitabine, and Zidovudine.

Non-limiting examples of other antiviral agents include Acemannan, Acetylleucine Monothanolamine, Amantadine, Amidinomycin, ATZ, Delavirdine, Foscarnet Sodium, Fuzeon, Indinavir, Interferon-α, Interferon-β, Interferon-γ, Kethoxal, Lysozyme, Methisazone, Moroxydine, Nevirapine, Podophyllotoxin, Ribavirin, Rimantadine, Ritonavir, Saquinavir, Stallimycin, Statolon, Tamiflu, Tromantadine, and Xenazoic Acid.

Non-limiting examples of anti-protozoan agents that may be used in the anti-infective compositions of the present invention include non-limiting examples of difluoromethylornithine (DFMO), CTP synthase inhibitors, benznidazole, chloroquine, amnio-quinolines, artemisinin, protease inhibitors like cruzipain, pentamidines, choline metabolism inhibitors, protein farnesyltransferase inhibitors, lanosterol 14-demethylase inhibitors, purine nucleoside phosphorylase inhibitors, miltefosine, and other purine metabolism enzyme inhibitors.

Compositions of the present invention are also useful to counteract the effect of prions. Prion is short for proteinaceous infectious particle that lacks nucleic acid (by analogy to virion) and is a type of infectious agent made only of protein. Prions are believed to infect and propagate by refolding abnormally into a structure that is able to convert normal molecules of the protein into the abnormally structured form, and they are generally quite resistant to denaturation by protease, heat, radiation, and formalin treatments, although potency or infectivity can be reduced. Qin, K. et al. Neuroscience (2006), 141(1), 1-8. The term does not, however, a priori preclude other mechanisms of transmission. The following diseases in animals are now believed to be caused by prions: scrapie in sheep, bovine spongiform encephalopathy (BSE), transmissible mink encephalopathy (TME), chronic wasting disease (CWD) in elk and mule deer, feline spongiform encephalopathy in cats, exotic ungulate encephalopathy (EUE) in nyala, oryx, and greater kudu. The following diseases in humans are believed to be caused by prions: several varieties of Creutzfeldt-Jakob Disease (CJD), such as Tatrogenic Creutzfeldt-Jakob disease, Variant Creutzfeldt-Jakob disease, Familial Creutzfeldt-Jakob disease, and Sporadic Creutzfeldt-Jakob disease; Gerstmann-Straussler-Scheinker syndrome (GSS), Fatal Familial Insomnia (FFI), Kuru, and Alpers syndrome.

A great deal of our knowledge of how prions work at a molecular level comes from detailed biochemical analysis of yeast prion proteins. A typical yeast prion protein contains a region (protein domain) with many repeats of the amino acids glutamine (Q) and asparagine (N); these Q/N-rich domains form the core of the prion's structure. Ordinarily, yeast prion domains are flexible and lack a defined structure. When the prion peptide convert to the prion state, several molecules of a particular protein come together to form a highly structured amyloid fiber. The end of the fiber acts as a template for the free protein molecules, causing the fiber to grow. Compounds of the present invention are capable of blocking amyloid plaque formation, including β-amyloid aggregation and assembly of aggregates on neuronal glycoproteins.

Non-limiting examples of at least one other disinfectant includes acid, alkali, alcohol, aldehyde, halogen, phenol, biguanide, peroxygen compound, quaternary ammonium compound, enzyme, amphoterics, surfactants, and combinations thereof.

Non-limiting examples of acids include acetic acid, phosphoric acid, citric acid, lactic, formic, and propionic acids, hydrochloric acid, sulfuric acid, and nitric acid.

Non-limiting examples of alkali include sodium hydroxide, potassium hydroxide, sodium carbonate, and ammonium hydroxide.

Non-limiting examples of alcohols include ethyl alcohol, isopropyl alcohol, and phenol.

Non-limiting examples of aldehydes include formaldehyde and glutaraldehyde.

Non-limiting examples of halogens include chlorine compounds such as hypochlorites, chlorine dioxide, sodium dichloroisocyanurate, and chloramine-T. Iodine compounds such as iodine and iodophors such as povidone-iodine.

Non-limiting examples of biguanides include chlorhexidine.

Non-limiting examples of peroxygen compounds include hydrogen peroxide and peracetic acid.

Non-limiting examples of QACs include benzalkonium chloride. Ethyl alcohol potentiates the action of QACs.

### Coatings

Coating refers to any temporary, semipermanent or permanent layer, covering or surface. Examples of coatings include polishes, surface cleaners, caulks, adhesives, finishes, paints, waxes polymerizable compositions (including phenolic resins, silicone polymers, chlorinated rubbers, coal tar and epoxy combinations, epoxy resin, polyamide resins, vinyl resins, elastomers, acrylate polymers, fluoropolymers, polyesters and polyurethanes, latex). Silicone resins, silicone polymers (e.g. RTV polymers) and silicone heat cured rubbers are suitable coatings for use in the invention and described for example in the Encyclopedia of Polymer Science and Engineering (1989) 15: 204 et seq. Coatings can be ablative or dissolvable, so that the dissolution rate of the matrix controls the rate at which the antiinfective agents are delivered to the surface. Coatings can also be non-ablative, and rely on diffusion principles to deliver the antiinfective agents to the surface. Non-ablative coatings can be porous or non-porous. A coating containing an antiinfective agent freely dispersed in a polymer binder is referred to as “monolithic” coating. Elasticity can be engineered into coatings to accommodate pliability, e.g. swelling or shrinkage, of the surface to be coated. The coating may also simply be an aqueous solution or suspension. In one embodiment, the coating is a silicone, polyurethane, resin, or aqueous coating.

### Disease Control in Livestock

The compositions of the present invention may be used in the treatment of livestock for the prevention of diseases. Despite advances in the development of chemotherapeutic drugs and effective animal vaccines, infectious disease remains a major issue for humans and animals. In addition to losses as a result of mortality, losses associated with infectious diseases in domestic animals arise from decreased productivity of meat, milk, or eggs, reproductive failure, and the cost of chemotherapy. Estimates of losses arising from infectious diseases vary from 15% to 20%.

Disinfection is an essential part of disease control programs for both endemic and exotic diseases. It is also used to minimize the risk of disease transmission between animals, including humans. With livestock, the minimization should not only be during the production phases but at the processing stage in meat plants and diaries. Thus, the composition of the present invention can be used to safely and effectively disinfect livestock, animal carcasses and equipment.

In one embodiment, the disease being prevented or treated is the H5N1 virus (also known as bird flu) in poultry, such as chickens. In a certain embodiments, the livestock or animal carcass, such as poultry, is sprayed with or dipped in a liquid or gaseous composition of the present invention. In other embodiments, the composition may be in a powder form for spraying or dipping livestock.

### Pharmaceutical and Personal Healthcare Formulations

The antiinfective compositions of the present invention may be administered by various means, depending on their intended use, as is well known in the art. For example, if compositions of the present invention are to be administered orally, they may be formulated as tablets, capsules, granules, powders or syrups. Alternatively, formulations of the present invention may be administered parenterally as injections (intravenous, intramuscular or subcutaneous), drop infusion preparations or suppositories. For application by the ophthalmic mucous membrane route, compositions of the present invention may be formulated as eye drops or eye ointments. These formulations may be prepared by conventional means, and, if desired, the compositions may be mixed with any conventional additive, such as an excipient, a binder, a disintegrating agent, a lubricant, a corrigent, a solubilizing agent, a suspension aid, an emulsifying agent or a coating agent.

In the aforementioned formulations, wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

Subject compositions may be suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of composition that may be combined with a carrier material to produce a single dose vary depending upon the subject being treated, and the particular mode of administration.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof: and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations which are suitable for vaginal administration also include pessaries, tampons, creams, gels, pastes, foams or spray formulations containing such carriers as are known in the art to be appropriate.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions of the present invention may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions.

Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

The dosage of any compositions of the present invention will vary depending on the symptoms, age and body weight of the patient, the nature and severity of the disorder to be treated or prevented, the route of administration, and the form of the subject composition. Any of the subject formulations may be administered in a single dose or in divided doses. Dosages for the compositions of the present invention may be readily determined by techniques known to those of skill in the art or as taught herein.

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 10 mg per kg.

An effective dose or amount, and any possible affects on the timing of administration of the formulation, may need to be identified for any particular composition of the present invention. This may be accomplished by routine experiment as described herein, using one or more groups of animals (preferably at least 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including composition, amounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The use of the subject compositions may reduce the required dosage for any individual agent contained in the compositions because the onset and duration of effect of the different agents may be complimentary.

Toxicity and therapeutic efficacy of subject compositions may be determined by standard pharmaceutical procedures in cell cultures or experimental animals, e.g., for determining the LD₅₀ and the ED₅₀.

The data obtained from the cell culture assays and animal studies may be used in formulating a range of dosage for use in humans. The dosage of any subject composition lies preferably within a range of circulating concentrations that include the ED₅₀ with little or no toxicity. The dosage may vary within this range depending upon the dosage form employed and the route of administration utilized. For compositions of the present invention, the therapeutically effective dose may be estimated initially from cell culture assays.

Applications include cosmetics and other over-the-counter products for human and animal application. Preservatives are used to prevent the growth of bacteria and fungi that may result in product contamination and deterioration. Compounds of the present invention can be used in combination with an existing preservative such as: alcohols; benzoic acid; chlorhexidine; diazolidinyl urea; dimethylol dimethylhydantoin-1,3-bis; isothiazolones; mercurials; parabens; phenolic compounds; quaternary ammonium compounds; and triclosan. Treatment concentrations could be reduced when these agents are used in combination with compounds of the present invention.

### Antimicrobial Surfaces

Certain naturally derived processed materials will be determined by artisans in these fields to especially suitable for the application or incorporation of compounds of the invention. A material can be contacted with the claimed compounds in a variety of ways including immersion and coating. In forms where the material has interstices, an antiinfective composition can reside therein as a liquid or as a gel. Fibrillar preparations can permit the fibers to be coated with the compound. Solid articles such as reconstructive blocks of hydroxyapatite can be painted with a coating of the compound for additional protection. These temporary means of application are appropriate for these materials because they only reside in the body temporarily, to be resorbed or replaced.

Implantable medical devices, using artificial materials alone or in combination with naturally-derived materials, can be treated with compounds either by surface coating or by incorporation. Metals may be suitably treated with surface coats while retaining their biological properties. In certain embodiments of the present invention, metals may be treated with paints or with adherent layers of polymers or ceramics that incorporate the compounds of the invention. Certain embodiments treated in this manner may be suitable for orthopedic applications, for example, pins, screws, plates or parts of artificial joints. Methods for surface treatment of metals for biological use are well-known in the relevant arts. Other materials besides metals can be treated with surface coats of compounds according to the present invention as the medical application requires.

Implantable devices may comprise materials suitable for the incorporation of the instant claimed compounds. Embodiments whose components incorporate compositions of the invention can include polymers, ceramics and other substances. Materials fabricated from artificial materials can also be destined for resorption when they are placed in the body. Such materials can be called bioabsorbable. As an example, polyglycolic acid polymers can be used to fabricate sutures and orthopedic devices. Those of ordinary skill in these arts will be familiar with techniques for incorporating agents into the polymers used to shape formed articles for medical applications. Antimicrobial compositions can also be incorporated into glues, cements or adhesives, or in other materials used to fix structures within the body or to adhere implants to a body structure. Examples include polymethylmethacrylate and its related compounds, used for the affixation of orthopedic and dental prostheses within the body. The presence of the compounds of the instant invention can decrease biofilm formation in those structures in contact with the glue, cement, or adhesive. Alternatively, a compound of the invention can coat or can permeate the formed article. In these compositions, the formed article allows diffusion of the compound, or functional portion thereof, into the surrounding environment, thereby preventing fouling of the appliance itself. Microcapsules bearing compounds can also be imbedded in the material. Materials incorporating compounds are adaptable to the manufacture of a wide range of medical devices, some of which are disclosed below. Other examples will be readily apparent to those practitioners of ordinary skill in the art.

In one embodiment, compounds of the invention can be applied to or incorporated in certain medical devices that are intended to be left in position permanently to replace or restore vital functions. As one example, ventriculoatrial or ventriculoperitoneal shunts are devised to prevent cerebrospinal fluid from collecting in the brain of patients whose normal drainage channels are impaired. As long as the shunt functions, fluid is prevented from accumulating in the brain and normal brain function can continue. If the shunt ceases to function, fluid accumulates and compresses the brain, with potentially life-threatening effect. If the shunt becomes infected, it causes an infection to enter the central portions of the brain, another life-threatening complication. These shunts commonly include a silicone elastomer or another polymer as part of their fabrication. Silicones are understood to be especially suited for combination with compounds according to the present invention.

Another shunt that has life-saving import is a dialysis shunt, a piece of polymeric tubing connecting an artery and a vein in the forearm to provide the kidney failure patient a means by which the dialysis equipment can cleanse the bloodstream. Even though this is a high-flow conduit, it is susceptible to the formation of biofilms and subsequent infection. If a shunt becomes infected, it requires removal and replacement. Since dialysis may be a lifelong process, and since there are a limited number of sites where shunts can be applied, it is desirable to avoid having to remove one through infectious complications. Imbedding or otherwise contacting the compounds of the invention with the shunt material can have this desired effect.

Heart valves comprising artificial material are understood to be vulnerable to the dangerous complication of prosthetic valve endocarditis. Once established, it carries a mortality rate as high as 70%. Biofilms are integrally involved in the development of this condition. At present, the only treatment for established contamination is high-dose antibiotic therapy and surgical removal of the device. The contaminated valve must be immediately replaced, since the heart cannot function without it. Because the new valve is being inserted in a recently contaminated area, it is common for prosthetic valve endocarditis to affect the replacement valve as well. Artificial heart valves comprised of the compounds of the invention may reduce the incidence of primary and recurrent prosthetic valve endocarditis. Compounds of the invention can be applied to the synthetic portions or the naturally-derived portions of heart valves.

Pacemakers and artificial implantable defibrillators commonly comprise metallic parts in combination with other synthetic materials. These devices, which may be coated with a polymeric substance such as silicone are typically implanted in subcutaneous or intramuscular locations with wires or other electrical devices extending intrathoracically or intravascularly. If the device becomes colonized with microorganisms and infected, it must be removed. A new device can be replaced in a different location, although there are a finite number of appropriate implantation sites on the body. Devices comprising the compounds of the invention may inhibit contamination and infection, or substantially reduce the risk thereof.

Devices implanted into the body either temporarily or permanently to pump pharmacological agents into the body can comprise metallic parts in combination with other synthetic materials. Such devices, termed infusion pumps, can be entirely implanted or can be partially implanted. The device may be partially or entirely covered with a polymeric substance, and may comprise other polymers used as conduits or tubes. Incorporating antiinfective compositions according to the present invention into the coating materials imposed upon these devices or into the materials used for the devices themselves, their conduits or their tubing may inhibit their contamination and infection.

Equally lifesaving are the various vascular grafting prostheses and stents intended to bypass blocked arteries or substitute for damaged arteries. Vascular grafting prostheses, made of Teflon, dacron, Gore-tex®, expanded polytetrafluoroethylene (e-PTFE), and related materials, are available for use on any major blood vessel in the body. Commonly, for example, vascular grafting prostheses are used to bypass vessels in the leg and are used to substitute for a damaged aorta. They are put in place by being sewn into the end or the side of a normal blood vessel upstream and downstream of the area to be bypassed or replaced, so that blood flows from a normal area into the vascular grafting prosthesis to be delivered to other normal blood vessels. Stents comprising metallic frames covered with vascular grafting prosthesis fabric are also available for endovascular application, to repair damaged blood vessels.

When a vascular grafting prosthesis becomes infected, it can spread infection through the entire bloodstream. Furthermore, the infection can weaken the attachment of the vascular grafting prosthesis to the normal blood vessel upstream or downstream, so that blood can leak out of it. If the attachment ruptures, there can be life-threatening hemorrhage. When a vascular grafting prosthesis becomes infected, it needs to be removed. It is especially dangerous to put another vascular grafting prosthesis in the same spot because of the risk of another infection, but there are often few other options. Vascular grafting prostheses comprising compounds of the invention can resist infections, thereby avoiding these devastating complications.

Vascular grafting prostheses of small caliber are particularly prone to clotting. A vascular grafting prosthesis comprising a compound of the invention may not only prevent biofilm formation, but also inhibit clotting as described above, allowing a smaller diameter vascular grafting prosthesis to be more reliable. A common site for clotting is the junction point between the vascular grafting prosthesis and the normal vessel, called the anastomosis. Even if an artificial vascular grafting prosthesis is not used, anywhere that two vessels are joined or anywhere there is a suture line that penetrates a natural blood vessel, there is a potential for clotting to take place. A clot in a vessel can occlude the vessel entirely or only partially; in the latter case, blood clots can be swept downstream, damaging local tissues. Using suture comprised of the compounds of the invention may inhibit clotting at these various suture lines. The smaller the vessel, the more likely that a clot forming within it will lead to a total occlusion of the vessel. This can have disastrous results: if the main vessel feeding a tissue or an organ becomes totally occluded, that structure loses its blood supply and can die. Microsurgery provides dramatic examples of the damage that can occur with anastomotic clotting. In microsurgery, typically only a single tiny vessel is feeding an entire tissue structure like a finger or a muscle. If the vessel clots off, the tissue structure can quickly die. Microsurgery typically involves vessels only one to four millimeters in diameter. It is understood that the sutures penetrating the vessel at the anastomosis are likely sites for clots to form. Microsurgical sutures comprising a compound of the invention would result in localized administration of an anticoagulant at the site most likely to be damaged by clotting.

Suture material used to anchor vascular grafting prostheses to normal blood vessels or to sew vessels or other structures together can also harbor infections. Sutures used for these purposes are commonly made of prolene, nylon or other monofilamentous nonabsorbable materials. An infection that begins at a suture line can extend to involve the vascular grafting prosthesis. Suture materials comprising a compound of the invention would have increased resistance to infection.

A suture comprising a compound of the invention would be useful in other areas besides the vasculature. Wound infections at surgical incisions may arise from microorganisms that lodge in suture materials placed at various levels to close the incision. General surgery uses both nonabsorbable and absorbable sutures. Materials for nonabsorbable sutures include prolene and nylon. Absorbable sutures include materials like treated catgut and polyglycolic acid. Absorbable sutures retain tensile strength for periods of time from days to months and are gradually resorbed by the body. Fabricating an absorbable or a nonabsorbable suture comprising a compound of the invention and which retains the handling and tensile characteristics of the material is within the skill of artisans in the field.

A general principle of surgery is that when a foreign object becomes infected, it most likely needs to be removed so that the infection can be controlled. So, for example, when sutures become infected, they may need to be surgically removed to allow the infection to be controlled. Any area where surgery is performed is susceptible to a wound infection. Wound infections can penetrate to deeper levels of the tissues to involve foreign material that has been used as part of the operation. As an example, hernias are commonly repaired by suturing a plastic screening material called mesh in the defect. A wound infection that extends to the area where the mesh has been placed can involve the mesh itself, requiring that the mesh be removed. Surgical meshes comprising a compound of the invention can have increased resistance to infection. Surgical meshes are made of substances like Gore-tex®, teflon, nylon and Marlex®. Surgical meshes are used to close deep wounds or to reinforce the enclosure of body cavities. Removing an infected mesh can leave an irreparable defect, with life-threatening consequences. Avoiding infection of these materials is of paramount importance in surgery. Materials used for meshes and related materials can be formulated to include the claimed compounds of the instant invention.

Materials similar to vascular grafting prostheses and surgical meshes are used in other sites in the body. Medical devices used in these locations similarly can benefit from the compounds of the invention; when these devices are located in the bloodstream, these agents' anticoagulant effects provide further benefit. Examples include hepatic shunts, vena caval filters and atrial septal defect patches, although other examples will be apparent to practitioners in these arts.

Certain implantable devices intended to restore structural stability to body parts can be advantageously treated with the instant claimed compounds. A few examples follow, and others will be readily identified by ordinary skilled artisans. Implantable devices, used to replace bones or joints or teeth, act as prostheses or substitutes for the normal structure present at that anatomic site. Metallics and ceramics are commonly used for orthopedic and dental prostheses. Implants may be anchored in place with cements like polymethylmethacrylate. Prosthetic joint surfaces can be fabricated from polymers such as silicones or Teflon™. Entire prosthetic joints for fingers, toes or wrists can be made from polymers.

Medical prostheses comprising compounds of the invention would be expected to have reduced contamination and subsequent local infection, thereby obviating or reducing the need to remove the implant with the attendant destruction of local tissues. Destruction of local tissues, especially bones and ligaments, can make the tissue bed less hospitable for supporting a replacement prosthesis. Furthermore, the presence of contaminating microorganisms in surrounding tissues makes recontamination of the replacement prosthesis easily possible. The effects of repeated contamination and infection of structural prosthetics is significant: major reconstructive surgery may be required to rehabilitate the area in the absence of the prosthesis, potentially including free bone transfers or joint fusions. Furthermore, there is no guarantee that these secondary reconstructive efforts will not meet with infectious complications as well. Major disability, with possible extremity amputation, is the outcome from contamination and infection of a structural prosthesis.

Certain implantable devices are intended to restore or enhance body contours for cosmetic or reconstructive applications. A well-known example of such a device is the breast implant, a gel or fluid containing sac made of a silicone elastomer. Other polymeric implants exist that are intended for permanent cosmetic or reconstructive uses. Solid silicone blocks or sheets can be inserted into contour defects. Other naturally occurring or synthetic biomaterials are available for similar applications. Craniofacial surgical reconstruction can involve implantable devices for restoring severely deformed facial contours in addition to the techniques used for restructuring natural bony contours. These devices, and other related devices well-known in the field, are suitable for coating with or impregnation with antiinfective compositions to reduce their risk of contamination, infection and subsequent removal.

Tissue expanders are sacs made of silicone elastomers adapted for gradual filling with a saline solution, whereby the filling process stretches the overlying tissues to generate an increased area of tissue that can be used for other reconstructive applications. Tissue expanders can be used, for example, to expand chest wall skin and muscle after mastectomy as a step towards breast reconstruction. Tissue expanders can also be used in reconstructing areas of significant skin loss in burn victims. A tissue expander is usually intended for temporary use: once the overlying tissues are adequately expanded, they are stretched to cover their intended defect. If a tissue expander is removed before the expanded tissues are transposed, though, all the expansion gained over time is lost and the tissues return nearly to their pre-expansion state. The most common reason for premature tissue expander removal is infection. These devices are subjected to repeated inflations of saline solution, introduced percutaneously into remote filling devices that communicate with the expander itself. Bacterial contamination of the device is thought to occur usually from the percutaneous inflation process. Once contamination is established and a biofilm forms, local infection is likely. Expander removal, with the annulment of the reconstructive effort, is needed to control the infection. A delay of a number of months is usually recommended before a new tissue expander can be inserted in the affected area. The silicone elastomer used for these devices is especially suitable for integrating with the antiinfective compositions of the present invention. Use of these agents in the manufacture of these articles may reduce the incidence of bacterial contamination, biofilm development and subsequent local infection.

Insertable devices include those objects made from synthetic materials applied to the body or partially inserted into the body through a natural or an artificial site of entry. Examples of articles applied to the body include contact lenses and stoma appliances. An artificial larynx is understood to be an insertable device in that it exists in the airway, partially exposed to the environment and partially affixed to the surrounding tissues. An endotracheal or tracheal tube, a gastrostomy tube or a catheter are examples of insertable devices partially existing within the body and partially exposed to the external environment. The endotracheal tube is passed through an existing natural orifice. The tracheal tube is passed through an artificially created orifice. Under any of these circumstances, the formation of biofilm on the device permits the ingress of microorganisms along the device from a more external anatomic area to a more internal anatomic area. The ascent of microorganisms to the more internal anatomic area commonly causes local and systemic infections.

As an example, biofilm formation on soft contact lenses is understood to be a risk factor for contact-lens associated corneal infection. The eye itself is vulnerable to infections due to biofilm production. Incorporating an antifouling agent in the contact lens itself and in the contact lens case can reduce the formation of biofilms, thereby reducing risk of infection. The antiinfective compositions of the present invention can also be incorporated in ophthalmic preparations that are periodically instilled in the eye.

As another example, biofilms are understood to be responsible for infections originating in tympanostomy tubes and in artificial larynxes. Biofilms further reside in tracheostomy tubes and in endotracheal tubes, permitting the incursion of pathogenic bacteria into the relatively sterile distal airways of the lung. These devices are adaptable to the incorporation or the topical application of antiinfective compositions to reduce biofilm formation and subsequent infectious complications.

As another example, a wide range of vascular catheters are fabricated for vascular access. Temporary intravenous catheters are placed distally, while central venous catheters are placed in the more proximal large veins. Catheter systems can include those installed percutaneously whose hubs are external to the body, and those whose access ports are buried beneath the skin. Examples of long-term central venous catheters include Hickman catheters and Port-a-caths. Catheters permit the infusion of fluids, nutrients and medications; they further can permit the withdrawal of blood for diagnostic studies or the transfusion of blood or blood products. They are prone to biofilm formation, increasingly so as they reside longer within a particular vein. Biofilm formation in a vascular access device can lead to the development of a blood-borne infection as planktonic organisms disseminate from the biofilm into the surrounding bloodstream. Further, biofilm formation can contribute to the occlusion of the device itself, rendering it non-functional. If the catheter is infected, or if the obstruction within it cannot be cleared, the catheter must be removed. Commonly, patients with these devices are afflicted with serious medical conditions. These patients are thus poorly able to tolerate the removal and replacement of the device. Furthermore, there are only a limited number of vascular access sites. A patient with repeated catheter placements can run out of locations where a new catheter can be easily and safely placed. Incorporation of antiinfective compositions within catheter materials or application of these agents to catheter materials can reduce fouling and biofilm formation, thereby contributing to prolonged patency of the devices and minimizing the risk of infectious complications.

As another example, a biliary drainage tube is used to drain bile from the biliary tree to the body's exterior if the normal biliary system is blocked or is recovering from a surgical manipulation. Drainage tubes can be made of plastics or other polymers. A biliary stent, commonly fabricated of a plastic material, can be inserted within a channel of the biliary tree to keep the duct open so that bile can pass through it. Biliary sludge which forms as a result of bacterial adherence and biofilm formation in the biliary system is a recognized cause of blockage of biliary stents. Pancreatic stents, placed to hold the pancreatic ducts open or to drain a pseudocyst of the pancreas, can also become blocked with sludge. Biofilms are furthermore implicated in the ascent of infections into the biliary tree along a biliary drainage tube. Ascending infections in the biliary tree can result in the dangerous infectious condition called cholangitis. Incorporation of compounds of the invention in the materials used to form biliary drainage tubes and biliary stents can reduce the formation of biofilms, thereby decreasing risk of occlusions and infections.

As another example, a peritoneal dialysis catheter is used to remove bodily wastes in patients with renal failure by using fluids instilled into and then removed from the peritoneal cavity. This form of dialysis is an alternative to hemodialysis for certain renal failure patients. Biofilm formation on the surfaces of the peritoneal dialysis catheter can contribute to blockage and infection. An infection entering the peritoneal cavity is termed a peritonitis, an especially dangerous type of infection. Peritoneal dialysis catheters, generally made of polymeric materials like polyethylene, can be coated with or impregnated with the antiinfective compositions to reduce the formation of biofilms.

As yet another example, a wide range of urological catheters function to provide drainage of the urinary system. These catheters can either enter the natural orifice of the urethra to drain the bladder, or they can be adapted for penetration of the urinary system through an iatrogenically created insertion site. Nephrostomy tubes and suprapubic tubes represent examples of the latter. Catheters can be positioned in the ureters on a semipermanent basis to hold the ureter open; such a catheter is called a ureteral stent. Urological catheters can be made from a variety of polymeric products. Latex and rubber tubes have been used, as have silicones. All catheters are susceptible to biofilm formation. This leads to the problem of ascending urinary tract infections, where the biofilm can spread proximally, carrying pathogenic organisms, or where the sessile organisms resident in the biofilm can propagate planktonic organisms that are capable of tissue and bloodstream invasion. Organisms in the urinary tract are commonly gram-negative bacteria capable of producing life-threatening bloodstream infections if they spread systemically. Infections wherein these organisms are restricted to the urinary tract can nonetheless be dangerous, accompanied by pain and high fever. Urinary tract infections can lead to kidney infections, called pyelonephritis, which can jeopardize the function of the kidney. Incorporating the antiinfective compositions can inhibit biofilm formation and may reduce the likelihood of these infectious complications.

A further complication encountered in urological catheters is encrustation, a process by which inorganic compounds comprising calcium, magnesium and phosphorous are deposited within the catheter lumen, thereby blocking it. These inorganic compounds are understood to arise from the actions of certain bacteria resident in biofilms on catheter surfaces. Reducing biofilm formation by the action of antiinfective compositions may contribute to reducing encrustation and subsequent blockage of urological catheters.

Other catheter-like devices exist that can be treated with antiinfective compositions. For example, surgical drains, chest tubes, hemovacs and the like can be advantageously treated with materials to impair biofilm formation. Other examples of such devices will be familiar to ordinary practitioners in these arts.

Materials applied to the body can advantageously employ the antiinfective compositions disclosed herein. Dressing materials can themselves incorporate the antiinfective compositions, as in a film or sheet to be applied directly to a skin surface. Additionally, antiinfective compositions of the instant invention can be incorporated in the glue or adhesive used to stick the dressing materials or appliance to the skin. Stoma adhesive or medical-grade glue may, for example, be formulated to include an antiinfective composition appropriate to the particular medical setting. Stoma adhesive is used to adhere stoma bags and similar appliances to the skin without traumatizing the skin excessively. The presence of infectious organisms in these appliances and on the surrounding skin makes these devices particularly appropriate for coating with antiinfective compositions, or for incorporating antiinfective compositions therein. Other affixation devices can be similarly treated. Bandages, adhesive tapes and clear plastic adherent sheets are further examples where the incorporation of an antiinfective composition in the glue or other adhesive used to affix the object, or incorporation of an antiinfective composition as a component of the object itself, may be beneficial in reducing skin irritation and infection.

A number of medical devices that are required to be sterilized prior to use can be adversely affected by the effects of heat, ethylene oxide, or electron beam irradiation processes that are typically employed in the practice of sterilization. These types of devices include endoscopic devices such as opthalmoscopes, and bioprocessing devices such as extracorporeal dialysis membranes used in hemodialysis applications. Some implantable devices, such as prosthetic heart valves, are similarly adversely affected by commonly used sterilization methods. Tissues used for transplantation can also be adversely affected by sterilization using heat, ethylene oxide, or electron beam irradiation processes.

Chemical sterilization, using biocides, is an accepted alternative for rendering otherwise labile materials sterile. Commonly used biocides for medical device and tissue sterilization include glutaraldehyde, formaldehyde, orthopthalaldehyde, and peracetic acid. When employed at sufficient concentrations and for sufficient contact times, these (and other) chemicals can render devices and tissues sterile.

Reducing chemical concentrations and contact times used in chemical sterilization processes improves device and tissue functionality, and provides an economic benefit to the manufacturer. Reduction of chemical concentrations can be achieved by forming synergistic compositions of the present invention where reduced amounts of chemical compounds achieve the same antiinfective effectiveness.

These above examples are offered to illustrate the multiplicity of applications of compounds of the invention in medical devices. Other examples will be readily envisioned by skilled artisans in these fields. The scope of the present invention is intended to encompass all those surfaces where the presence of fouling has adverse health-related consequences. The examples given above represent embodiments where the technologies of the present invention are understood to be applicable. Other embodiments will be apparent to practitioners of these and related arts. Embodiments of the present invention can be compatible for combination with currently employed antiseptic regimens to enhance their antiinfective efficacy or cost-effective use. Selection of an appropriate vehicle for bearing a compound of the invention will be determined by the characteristics of the particular medical use. Other examples of applications in medical environments to promote antisepsis will be readily envisioned by those of ordinary skill in the relevant arts.

Yet another example includes the use of the flavononols and luecoanthocyanidins as a design platform and/or scaffold for the development of vaccines. Since the compounds of the invention bind to a surface or hemagglutinin binding site of influenza viruses, they can provide design and structural requirements for universal influenza vaccine development. This peptide or modified forms known in the art can be used to create vaccines that will lead to antibodies that will inactivate the initial infection step of influenza viruses.

### Crop Protection

Compositions of the present invention may also be used to form antiinfective surfaces on plants. Plants refers to any member of the plant kingdom, at any stage of its life cycle, including seeds, germinated seeds, seedlings, or mature plants. Plant cells refer to a cell from a plant or plant component. Plant component refers to a portion or part of a plant. Examples include: seeds, roots, stems, vascular systems, fruits (further including pip fruits, e.g. apples, pears, quinces), citrus fruits (oranges, lemons, limes, grapefruits, mandarins, nectarines), stone fruits (peaches apricots, plums, cherries, avocados, grapes), berries (strawberries, blueberries, raspberries, blackberries), leaves, grains and vegetables. The compositions of the present invention are effective at protecting plants from various organisms that infect plants or plant components. Examples include molds, fungi and rot that typically use spores to infect plants or plant components (e.g. fruits, vegetables, grains, stems, roots). Spores must recognize the host, attach, germinate, penetrate host tissues, and proliferate by hyphae that will allow the fungus to access to nutrients from the plant for growth and reproduction.

In addition to antibiotics such as streptomycin and tetracycline, which are used for treating some bacterial infections of plants, typical antifungal treatments that could be used in combination with the compounds of the present invention include: acetylanilines such as metalazyl; benzimidazoles such as benomyl/MBC; chlorinated nitrobenzenes such as tetrachloronitrobenzene; chloroneb; chlorothalonil; dinitro derivatives such as dinitro-o-cresol; dodine; fenaminosulf, fenarimol and other sterol inhibitors; heavy metals such as copper; heterocyclic nitrogen compounds such as glyodin; oxathiins such as carboxin; quinones such as cloranil; sulfur and sulfur-containing compounds such as dithiocarbamates; terrazole; and tricyclazole. Treatment concentrations and/or contact times could be reduced when these agents are used in combination with compounds of the present invention.

### Food Production and Processing

Compositions of the present invention may also be used to form antiinfective surfaces on equipment and clothing generally used in the food processing or production fields. Compositions of the present invention may be applied by spraying, using a high-pressure washer set at low pressure or, for small areas, a knapsack sprayer.

Disinfection of transport vehicles may prove difficult because of their construction, presence of uneven surfaces, and cold ambient temperatures (Böhm R., 1999). High pressure cleaning with warm water containing the disinfectants of the present invention may be followed by rinsing with hot water. When surfaces are dry, disinfectant at the correct concentration should be applied by spraying all parts of the vehicle, including the bodywork and wheels, and left to act for at least 30 minutes. The interior of the driver's compartment, especially the floor, should be cleaned and disinfected also.

Contaminated footwear may transfer infectious agents from one location to another, especially pathogens shed in feces or urine. Footbaths should be used by all staff and visitors. Unless all personnel wear waterproof footwear, footbaths will not contribute to disease prevention.

Footbaths comprising compositions of the present invention should be changed frequently and the date of change should be recorded. If used constantly on a large farm or unit, the composition should be changed daily or more frequently if there is evidence of gross contamination. Replacement of the composition at 3-day intervals may suffice on smaller units. If gross soiling of footwear is unavoidable, a second footbath with diluted detergent should be placed alongside the footbath for washing of footwear before immersion in disinfectant.

Brief immersion of footwear in a footbath may not be satisfactory as a disease control measure. Immersion of clean footwear to a depth of about 15 cm in an effective amount of the disinfectant composition of the present invention for at least 1 minute is a minimum requirement. Footbaths, located at suitable entry points to a farm or building, should be protected from flooding by surface water or rainfall. Antifreeze compatible with the disinfectant composition may be added in frosty weather. Alternatively, footbaths may be moved indoors at entry points to avoid freezing.

Vehicles visiting farms in succession may occasionally transfer infectious agents on the body of the vehicle or on its wheels. Wheel baths are sometimes used at farm entrances as part of a disease control program.

The design construction and use of wheel baths should ensure that there is adequate contact with the compositions of the present invention for a sufficient time to ensure destruction of infectious agents on the surface of the wheels. The site for installation of a wheel bath should be carefully selected to minimize the risk of flooding, contamination by surface water, or subsidence. The dimensions of the bath should ensure accommodation of the largest vehicles entering the farm. The tire of the largest wheel entering the bath should be completely immersed in disinfectant in one complete revolution.

Wheel baths, which should be built to high specifications, should be waterproof and free of structural defects. No valves or openings that might allow accidental pollution of water courses should be included in the design. The capacity of the bath should allow for heavy rainfall or snowfall without the risk of disinfectant overflow. A depth gauge could be incorporated into the design to indicate dilution or evaporation of disinfectant.

The intervals between changing are important considerations. An advantage of the present compositions is their stability which means they need not be changed as frequently as with other antiinfective compositions. If wheels have caked organic matter or grease on their surfaces, a wheel bath may have minimal effect.

Transfer of infectious agents from one premise to another on the wheels of vehicles, although possible, is relatively unimportant compared with other sources of infection. The contents of vehicles, including animals and their secretions and excretions, animal feed, and the clothing and footwear of drivers and passengers pose a much greater threat to healthy animals than vehicle wheels.

### Antifungal and Antiprotozoan Application

Typical treatments that could be used in combination with the compounds of the present invention include: antibiotics such as ivermectin for nematodes; antimony compounds such as lithium antimony thiomalate for Leishmania spp.; atabrine compounds such as quinacrine HCl for malaria (Plasmodium spp. and others); benzimidazole carbamates such as albendazole for GI nematodes; bephenium/thenium compounds such as bephenium hydroxynaphthoate for intestinal nematodes; bisphenols such as bithonol for tapeworms; chorinated hydrocarbons such as tetrachloroethylene for hookworms; chloroquines such as aralen for malaria (Plasmodium spp. and others); cyanine dyes such as pyrvinium pamoate for pinworms; diamidines such as stillbamidine for Leishmania spp.; diodoquin for amoebae and Giardia spp.; imidazothiazoles such as levamisole for lung worm and GI nematodes; nitroimidazoles such as metronidazole for trichomonads and amoebae; niclosamides such as bayluscide for tape worm; niridazole for schistosomes; organophosphates such as trichlorphon for GI nematodes; phenothiazine for GI nematodes; piperazines such as diethylcarbamaine for Ascarid and filarial nematodes; sulfonamides such as sulfadimidine for malaria (Plasmodium spp. and others); and suramin for trypanosomes. Treatment concentrations and/or contact times can be reduced when these agents are used in combination with the compounds of the present invention.

### Diagnostics and Biosensors

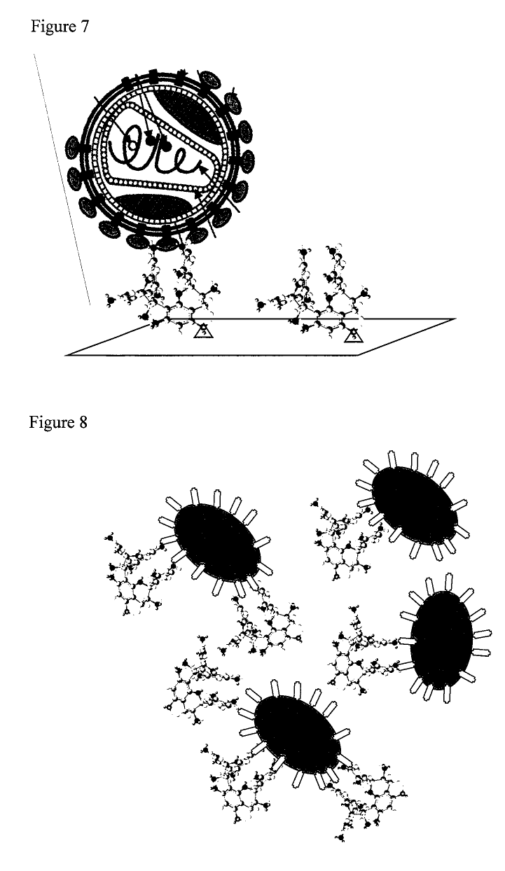

In another aspect of the invention, the aforementioned compounds can be used as diagnostics agents. In particular, the compounds may used as biosensors. For example, a tethered form of the pharmaceutical compositions can be used for detection, identification, decontamination and protection from infectious bacterial, fungal, viral and prion agents and non-infectious amyloid agents (FIG. 7). The chemical tether, such as an ester or amide linkage to the A ring of the monomer of the pharmaceutical compositions here are shown as A. The tether is preferred on the A ring so that the active binding domain defined by the two phenolic rings of Rings B and C are free to interact with binding motifs on the targeted pathogens.

In another embodiment, a solution form of the pharmaceutical compositions can be used for detection, identification, decontamination and protection from infectious bacterial, fungal, viral and prion agents and non-infectious amyloid agents (FIG. 8). The active phenolic binding domains of Rings B and C of the pharmaceutical compositions here interaction with binding motifs on the targeted pathogens.

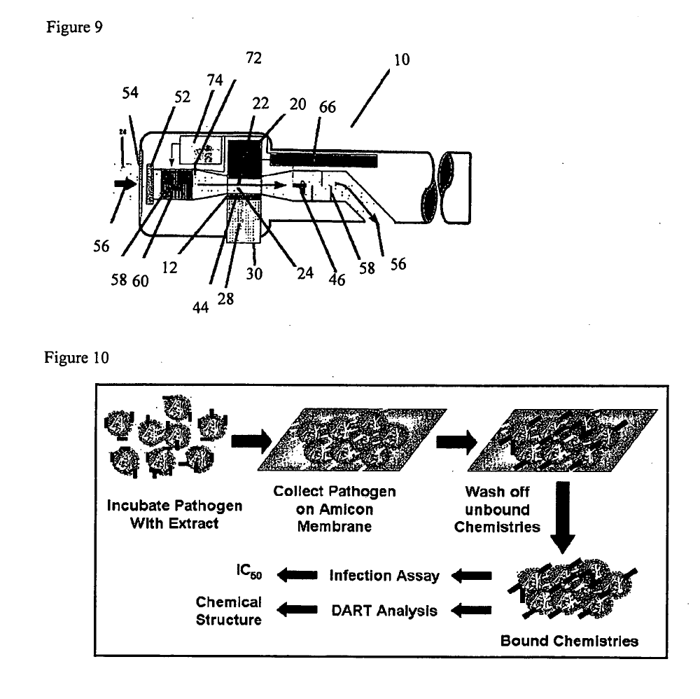

In another embodiment, a comprising the compounds of the present invention can be used device for detection/identification of infectious agents and amyloid agents in an aqueous environment or vapor phase environment (FIG. 9). The device include a means of collected the sample stream, interrogating that stream with a solid support film on which the pharmaceutical compositions here are tethered and available for binding targeted ligands—pathogens or amyloids, and for which the binding event reports the detection/identification of said target through an optical or other physical signal that reports the recognition event.

### EXEMPLIFICATION

Identification of compounds from botanicals: A botanical extract (powder, paste or liquid) is lyophilized and subject to a warm water (40° C.) extract and enhanced supercritical CO₂ extraction procedure and affinity chromatography using methods described (Li D, Gow R T, Sypert, G W: Methods and compositions comprising Elder species. 2006. PCT/US07/064,286). To obtain compositions of the present invention the lyophilized material can be extracted with warm water (40° C.) and the eluate is loaded onto Celite, and the pellet is discarded. The Celite-bound material is washed with low ionic strength Tris-HCl buffer (pH 8.2), and the washed material discarded. The Celite-bound fraction is released with high ionic strength K-phosphate buffer, collected and then loaded onto hydroxyapatite. The fractions of interest, flavonol, flavononol and proanthocyanidin are collected with an increasing gradient of K-phosphate buffer, and the lower molecular weight (<250 MW) phenolic fraction is discarded.

Chemical Characterizations: Time-of-flight mass spectrometry was used to further characterize the compositions of the present invention. The JEOL DART AccuTOF-DART-D mass spectrometer (JMS-T 100LC; Jeol USA, Peabody, Mass.) technology used here requires no sample preparation and yields masses with accuracies to 0.0001 mass units (Cody R B, Laramée J A, Nilles J M, Durst H D: Direct Analysis in Real Time (DART™) Mass Spectrometry. JEOL News 2005, 40:8-12). For positive ion mode (DART+), the needle voltage was set to 3500V, heating element to 300° C., electrode 1 to 150V, electrode 2 to 250V, and helium gas flow to 3.98 liters per minute. For the mass spectrometer, the following settings were loaded: orifice 1 set to 20V, ring lens voltage set to 5V, and orifice 2 set to 5V. The peak voltage was set to 1000V in order to give peak resolution beginning at 100 m/z. The microchannel plate detector (MCP) voltage was set at 2550V. Calibrations were performed internally with each sample using a 10% (w/v) solution of PEG that provided mass markers throughout the required mass range 100-1000 m/z. Calibration tolerances were held to 5 mmu. Samples (as dry powders) of the composition of the present invention were introduced into the DART helium plasma using the closed end of a borosilicate glass melting point capillary tube held in the He plasma for approximately 3-5 seconds per analysis. No pyrolysis of samples was observed during the analyses.

Molecular formulas and chemical structures were identified and confirmed by elemental composition and isotope matching programs in the Jeol MassCenterMain Suite software (MassCenter Main, Version 1.3.0.0; JEOL USA Inc.: Peabody, Mass., USA, Copyright® 2001-2004). A searchable database of flavonoid structures and masses was developed using an existing database (Cook N C, Samman S: Flavonoids—Chemistry, metabolism, cardioprotective effects, and dietary sources. J Nutr Biochem 1996, 7:66-76) and one developed by HerbalScience for natural products. In addition, molecular identification were searched and verified against the NIST/NIH/EPA Mass Spec Database when needed (Stein S, Mirokhin Y, Tchekhovskoi D, Mallard G, Mikaia A, Zaikin V, Little J, Zhu D, Clifton C, Sparkman D: The NIST mass spectral search program for the NIST/EPA/NIH mass spectral library—Version 2.0d. National Institute of Standards and Technology, Gaithersburg, Md., 2005). All chemical identifications in the mass spectra were assigned with a confidence level greater than 90%.

Influenza Viruses and Cells: Purified human Influenza A/PR/8/34 (H1N1) virus was obtained from Advanced Biotechnologies Incorporated and used directly without further passage. Avian influenza A virus reassortant Indo/o5/2005(H5N1)/P8-IBCDC-RG2 reference strain was obtained from the CDC. Madin-Darby canine kidney NBL-2 (MDCK) cells were purchased from the American Type Culture Collection and were grown in Dulbecco's modified Eagle's medium (DMEM) supplemented with 10% (v/v) fetal bovine serum (FBS), 2 mM glutamax, 100 U/ml penicillin G and 100 mg/ml streptomycin, (Invitrogen) at 37° C. with 5% (v/v) CO₂. The MDCK cells were used for all influenza virus infection studies.

Influenza Viral Focus-forming Inhibition Assays: Target MDCK cells were seeded at a density of 3×10⁵ cells per well in 6-well plates 24 h prior to infection. Extracts were dissolved in a minimal volume of 1% (v/v) ethanol (USP) prior to dissolving in phosphate buffered saline (PBS; pH 7.2) (Invitrogen) and the soluble fraction was buffered to pH 7.2 with HEPES (pH 7.2) and NaOH. Approximately 200 focus-forming units (FFU) of influenza virus were incubated with or without two-fold dilutions of extracts in PBS for DMEM for 1 h at room temperature. Virus/extract or virus/control antibody mixtures were allowed to infect confluent MDCK monolayers for 30 min at room temperature, after which time the medium was removed and the cells were overlaid with fresh DMEM containing 0.85% (w/v) Sea-Plaque agarose (Cambrex BioScience), 0.288% (v/v) bovine serum albumin, 2 mM glutamax, and 0.096% trypsin (w/v; 1 mg/ml) (Worthington Biochemical Co.). Infected cells were incubated at 37° C. with 5% (v/v) CO₂ for 27 h. Cultures were fixed with 10% (w/v) formalin solution (Formalde-fresh (Fisher Scientific) overnight at 4° C. and permeablized with 70% (v/v) ethanol (USP) prior to immunostaining and visualization using goat anti-influenza A virus IgG polyclonal antibody (Chemicon) followed by a rabbit Anti-Goat IgG (H & L) horseradish peroxidase conjugated affinity purified antibody (Chemicon) and AEC chromogen substrate (Dako).

Inhibition of human Influenza H1N1 virus infection in vitro: A focus-forming assay was used to characterize the anti-influenza virus activity of the compounds of the present invention. Human influenza A virus subtype /PR/8/34 H1N1 were pre-incubated for 1 hour with two-fold serial dilutions of extract prior to delivery to target MDCK cell cultures. Virus infection was visualized in MDCK target cells using an antibody coupled calorimetric reaction. All extracts were buffered to pH 7.0-7.2 with HEPES buffer (pH 7.2) prior to use in focus-forming assays to ensure that viral inhibitory effects were not due to a pH-triggered inactivating conformational change in the virus. The buffer conditions did not inhibit virus entry in control experiments. Infectious events were scored over a concentration range of compounds to generate viral infection inhibition curves, and IC₅₀ and IC₁₀₀ values for the different compounds. All compounds generated dose-dependent inhibition curves. The concentration of extract at which 50% of the virus was inhibited (IC₅₀) and the 100% inhibition level (IC₁₀₀) values were determined from mathematical analyses derive from the curve fitting program. The IC₅₀ value was 252±35 (±1 SD) μg/ml while the IC₁₀₀ value was 1,108 μg/ml±81 (±1 SD). Importantly, the compounds showed 100% inhibition of viral entry. Inhibition data is summarized in Table 1.

Inhibition of Avian Influenza A (H5N1) virus infection in vitro: The focus-forming assay was used also to characterize the activity of compound of the present invention against avian flu. Avian influenza A virus reassortant Indo/05/2005(H5N1)/P8-IBCDC-RG2 reference strain was treated as described for the H1N1 viruses. A dose-dependent inhibition of H5N1 infection was obtained and data are summarized in Table 1. The IC₅₀ value was 412±20 (±1 SD) μg/ml while the IC₁₀₀ value was 7414 μg/ml±1159 (±1 SD). Again, the compounds of the present invention showed 100% inhibition of H5N1 viral entry.

Microbial Adhesion Assays: Bacterial and fungal strains were grown at 37° C. in appropriate media in liquid culture to ca. 10⁴ mL, and an aliquot was subcultured and fresh media, 24 hr prior to the initiation of the adhesion assays. Approx. 0.5 OD of bacteria or fungi were diluted in PBS to yield 10³-10⁴ cells/ml, and cell were added to 96 well plates that contained serially diluted concentrations of the elderberry extract HSS-351. Bacteria or fungi were incubated at 37 C with gently shaking in Tecan GenosisPro microplate reader for 20-30 min to allow for adhesion of bacterial cells. Plates were then washed with a Tecan plate washer three times to remove unbound and weakly bound cells. The cells are fixed with 10% (v/v) ethanol (USP) and stained with SYTO 13 (Molecular Probes) which stains DNA. Cells are counted by monitoring fluorescence at 485 nm excitation and 525 nm emission using the BioTek Synergy 4 microplate reader.

TABLE 1
Infection inhibition of influenza and adhesion inhibition of bacteria
and fungus with a purified compound of the present invention as well
as an extract containing compounds of the present invention.

IC₅₀ value

| | Compound | Extract |
|---|---|---|
| Pathogen | (μM) | (μg/mL) |
| Influenza A (H1N1) | 5.43 | 252 |
| Avian Influenza (H5N1) | ND | 412 |
| Candida albicans (ATCC# 96133) | 89.1 | 0.98 |
| Escherichea coli (ATCC# 53499) | 60.0 | 1.21 |

ND = not determined.

Direct Binding Assay for Influenza Viruses: Through the use of the Direct Binding Assay and DART fingerprinting, it was possible to determine which compounds were binding to the H1N1 virus particles. FIG. 1 show the DART positive ion fingerprints of the compounds bound to H1N1 (FIG. 1B) and those chemistries that are washed off the virions (FIG. 1A) and, therefore, do not bind. The dominant compounds that bind to the H1N1 viral particles are certain flavonoids of the present invention (FIG. 1B). The nature and chemical characterization of the bound compounds is provided below.

In a similar manner we examined the Avian flu H5N1 virus using the direct binding assay to determine the compounds that bind to this virus (FIG. 2). Again as with H1N1, the dominant chemistries that bind to the H5N1 particles are flavonoids compounds of the present invention (FIG. 2B). The nature and chemical characterization of the bound chemistries is provided below. Other compounds (phenols, phenolic acids and most of the flavonoids) were found not to bind to H5N1 virions.

Direct Binding Assay: A Direct Binding Assay (DBA) was developed to determine which of the bioactive compounds in an extract and the compositions of the present invention function to inhibit influenza infection. The assay involved the incubation of the target virus or bacterium in the buffered (pH 7.2-7.4) extract for 1 h, after which the viruses were filtered onto an Amicon 100K Da molecular filter which retained the virions or bacteria, but allowed the unbound compounds to be removed. The viruses or bacteria are washed on the membrane twice with PBS (pH 7.2) which effectively removed unbound compounds. The virus particles or the bacterial cells were then collected and a small portion fixed in 100% (USP) ethanol for DART TOF-MS analyses. The remaining portion of virus particles or bacterial cells with bound compounds were used for either viral focus forming infection inhibition assays as described above or for bacterial adhesion assays.

Re-infection Assays for Viruses. The Direct Binding Assay (see above) was used to validate the specific role and mode-of-action of compounds of the present invention. The H1N1 virus particles were incubated at the corresponding IC₅₀ and IC₁₀₀ concentrations for 1 h. Following the Direct Binding Assay described above, the H1N1 viruses with bound compounds of the present invention and washed free of any unbound compounds were subjected to the same infection assay as used for the initial infection studies. The data revealed that when the virus compounds of the present invention are bound to the viruses nearly the binding sites on the virus are occupied stoichiometrically, as evidenced by the percent inhibition achieved (80% and 20%, respectively for the IC₁₀₀ and IC₅₀ incubations) when the viruses from the DBA were allowed to infect MDCK cells. During the DBA the virions lose some viability which likely accounts for the differences between percent inhibition achieved for the H1N1 infection post-DBA and the anticipated 100% and 50% inhibition expected due to the incubation concentration.

Cell Target Cytotoxicity Assays: To verify that the viral inhibitory effects were not due to extract- or compound-induced cellular toxicity, by the compounds of the present invention the extract was tested using a standard MTT colorimetric cell viability assay. No statistically significant cellular toxicity was observed over the concentration range that inhibited virus infection in vitro. The cytotoxicity of extracts or the pharmaceutical compositions herein was measured by monitoring mitochondrial reductase activity in MDCK cells using the TACS™ MTT cell proliferation assay (R&D Systems, Inc.) according to the manufacturer's instructions. Two-fold serial dilutions of buffered extracts in PBS were added to MDCK cells in a 96-well plate and incubated at 37° C. with 5% (v/v) CO₂ for 48 h. Absorbance at 560 nm was measured using a Tecan GeniosPro plate reader (Tecan US).

DART TOF-MS analysis of Viral Bound Compounds: The fractions containing the viruses and bound compounds were analyzed using a DART AccuTOF mass spectrometer (Jeol USA, Peabody, Mass.). The setting for the DART ionization source were: needle voltage=3500V, temperature=300° C., Electrode 1=150V, Electrode 2=250V, and helium gas flow=3.49-3.89 LPM. For the mass spectrometer, the following settings were loaded: Orifice 1=20V, Ring Lens voltage=5V, and Orifice 2=5V, the peaks voltage=1000V, the microchannel plate detector (MCP)=2550V. Calibrations were performed internally with each sample using a 10% solution of PEG 600 (Ultra Chemicals, North Kingston R.I.) providing mass markers throughout the required mass range of 100-800 amu. Samples were introduced into the He plasma after resuspension in PBS described above using the closed end of a borosilicate glass melting point capillary tube. The capillary tube was held in the He plasma until signal disappeared from the total-ion-chromatogram (TIC) and the signal to noise ration (S/N) returned to baseline values.

Candidate molecular formulae were identified using elemental composition and isotope matching programs in the Jeol MassCenterMain Suite software (JEOL USA, Peabody, Mass.). The candidate molecular formulae were assigned with a confidence level greater than 90%. These candidate molecular formulae were used, in conjunction with mass spectrometric fragment analysis and molecular modeling, to determine chemical structures. The compound identified, at m/z [M+H]⁺=479.232 is most likely esterified with 3,4,5-trihydroxy-cyclohexanecarboxylic acid, but may also be glycosylated with dihydroxy-methyltetrahydropyran carboxylic acid on the 3-OH of the flavononol C-ring (FIG. 4). Upon further evaluation of the mass spectral data, it was determined that the peak at m/z=214.089 represents the [M••] radical occurring from the DART-generated fragmentation of the C-ring of the aglycone of Tristenonol (F. Cuyckens and M. Claeys, 2004. Mass spectrometry in the structural analysis of flavonoids. Journal of Mass Spectrometry. 39:1-15).

Summary of Viral Direct Binding Data: In Table 2 the binding ratios and relative percent of total binding species of the compositions of the present invention (e.g., Tristenonol) and other flavonoids derived from botanical extracts are summarized. A direct binding assay (DBA) was conducted on envelope viruses including H1N1, H5N1, DNV-2, and HIV-1 as well as the non-envelope Rhinovirus (HRV-16), and it was shown through the DBA that the compounds of the present invention bind specifically to influenza viruses. The percent of the flavononols of the present invention that bind to influenza viruses ranges from ca. 7 to ca. 27% depending on the hemagluttinin type (H1 vs. H5) on the surface of the influenza virus and that these compounds represent a significant portion of the species that bind to the influenza viruses. For all influenza types examined, the ratios of bound anti-infective flavononols (Table 2) are significantly different from their abundances in the original botanical in which the viruses were incubated, indicating the binding interactions are specific for influenza viruses and not simply driven by mass action.

TABLE 2
Ratios from the direct binding assay of influenza viruses
using an extract containing compounds of the present invention.
Percentages were determined based on relative abundances
of all viral bound chemicals after conducting the Direct
Binding Assay (DBA) as described above.

| | Percent (%) of flavononol |
|---|---|
| | contribution to total bound |
| | compounds following the DBA |
| Influenza A virus (H1N1) | 26.6 |
| Avian Influenza virus (H5N1) | 6.9 |
| Extract composition | 37.6 |

Microbial and Amyloid Direct Binding Assays: A Direct Binding Assay was used to determine which of the bioactive chemistries in the botanical extracts or pharmaceutical compositions herein bind to the different microbes (Gram positive and Gram negative bacteria, fungi, prions, amyloids). The microbe or amyloids were incubated in the pharmaceutical composition or extract for 1 h, filtered onto Amicon 100K Da cutoff membranes which retained the virions, and washed twice with PBS (pH 7.2) which effectively removed unbound chemistries. The microbes or amyloids were then collected and a small portion fixed in 100% (USP) ethanol to kill and fix the particles for DART TOF-MS analyses while the remaining particles with bound chemistries were used for adhesion assays or amyloid aggregation assays. Inactivated microbial particles were resuspended in PBS prior to DART TOF-MS positive ion analyses.

Viral and Bacterial Vaccine Applications: The compounds of the invention can be used to develop vaccines. For example, the compounds of the present invention bind to specific amino acid motifs within the “adhesin’ domain on the envelope or capsid of viruses, on the pilin adhesins of Gram negative bacteria or the mini-pilin adhesin domain on Gram positive bacteria. Based on molecular modeling these amino acid motifs are 3-7 amino depending on 3-D structure abut at restricted to a size of <10-12 Å, which is based on the folding of the binding domains of the compounds of the present invention.

### Synthesis of the Flavononol Aglycone:

2,4,6-Tris(methoxymethoxy)acetophenone: A mixture of 2,4,6-trihydroxyacetophenone (1.0 g, 5.37 mmol) in dry DMF (20 mL) was added to a slurry of sodium hydride (60% in mineral oil, 0.86 g, 20 mmol) in dry DMF (10 mL) at 0-5° C. over period of 0.5 h under N₂ and stirred for 1.0 h at RT. The reaction mixture was again cooled to 0-5° C.; a solution of chloromethyl methylether (1.75 g, 21.7 mmol) in dry DMF was added slowly over a period of 0.25 h. The reaction mixture was stirred at RT for 4.0 h and poured in to ice-cold water (100 mL), extracted with ethyl acetate (2×50 mL). The combined organic layer was washed with water (50 mL), brine (50 mL) and dried over Na₂SO₄. The filtered organic layer was concentrated under vacuum and the resultant oily residue was purified by column chromatography (Column dimensions: 12″/0.7″ L/W, Silica gel: 230-400 mesh) by eluting with hexanes/ethyl acetate (9:1) followed by hexanes/ethyl acetate (8.5:15) to give compound 2,4,6-Tris(methoxymethoxy)acetophenone (0.78 g, 48%).

3,4,5-Tris(methoxymethoxy)benzaldehyde: A mixture of 3,4,5-trihydroxy benzaldehyde.H₂O (0.5 g, 2.9 mmol), potassium carbonate (4.0 g, 29.0 mmol), and dry acetone (100 mL) were placed in a 2 necked RB flask under N₂ and the mixture was cooled to 10-15° C. Chloromethyl methylether (1.436 g, 17.8 mmol) was added slowly over a period of 0.5 h at 10-15° C. and the reaction mass was allowed to reflux slowly over a period of 1.0 h. After refluxing for 6.0 h, the reaction mixture was filtered, washed with acetone (50 mL), concentrated in vacuum and extracted with ethyl acetate (2×25 mL). The combined organic layer was washed with water (25 mL), brine (25 mL) and dried over Na₂SO₄. The filtered organic layer was concentrated and the resultant oily residue was purified by column chromatography (Column dimensions: 12″/0.7″ L/W, Silica gel: 230-400 mesh) by eluting with hexanes/ethyl acetate (8:2) to give 3,4,5-Tris(methoxymethoxy)benzaldehyde (0.6 g, 72%).

3,4,5,2,4,6-Hexakis(methoxymethoxy)chalcone: To a mixture of 2,4,6-Tris (methoxymethoxy)acetophenone (1.0 g, 3.33 mmol) in absolute ethanol (5 mL) was added a solution of 40% potassium hydroxide in ethanol (20 mL) below 20° C. After stirring for 0.25 h, a solution of 3,4,5-Tris(methoxymethoxy)benzaldehyde (1.0 g, 3.5 mmol) in absolute ethanol (5.0 mL) was added slowly over a period of 10 min and allowed to stir overnight at RT. The reaction mass was quenched with water (50 mL) and extracted with ethyl acetate (2×50 mL). The combined organic layer was washed with water (50 mL), brine (50 mL) and dried over Na₂SO₄. The organic layer was concentrated under vacuum to give compound 3,4,5,2,4,6-Hexakis (methoxymethoxy) chalcone as a pale yellow solid (1.5 g, 78%).

3-(3,4,5-Tris-methoxymethoxy phenyl)-1-(2,4,6-tris-methoxymethoxy phenyl)-propenone: Hydrogen peroxide (50%, 1.0 mL, 17.35 mmol) was added to a mixture of chalcone 3,4,5,2,4,6-Hexakis (methoxymethoxy) chalcone (1.0 g, 1.76 mmol), sodium hydroxide (2N, 3.0 mL) in methanol (30 mL) at 15-20° C. and the reaction mixture was stirred for overnight at RT. The methanol was concentrated under vacuum and the resultant residue was extracted with ethyl acetate (2×50 mL). The combined organic layer was washed with water (50 mL), brine (50 mL) and dried over Na₂SO₄. The organic layer was concentrated under vacuum to give compound 3-(3,4,5-Tris-methoxymethoxy phenyl)-1-(2,4,6-tris-methoxymethoxy phenyl)-propenone as thick pale yellow oil. (0.72 g, 70%)

3,5,7-Trihydroxy-2-(3,4,5-trihydroxy phenyl)-chroman-4-one: A mixture of 3-(3,4,5-Tris-methoxymethoxy phenyl)-1-(2,4,6-tris-methoxymethoxy phenyl)-propenone (0.2 g) and HCl/absolute MeOH (1.25 M, 3.0 ml, 3.75 mmol) in absolute methanol (3.0 mL) was stirred at 45° C. for 0.5 h. The methanol was concentrated under vacuum and the resultant dark residue was purified by column chromatography (Column dimensions: 16″/0.5″ L/W, Silica gel: 230-400 mesh) by eluting with ethyl acetate/hexanes (1:1, 200 mL) followed by dichloromethane/methanol (9:1, 100 mL) to give compound 3,5,7-Trihydroxy-2-(3,4,5-trihydroxy phenyl)-chroman-4-one (0.70 g, 66%). ¹H NMR (d₆-acetone; 400 MHz) δ 6.62 (2H, s), 5.98 (1H, d, J=12 Hz), 5.94 (1H, d, J=12 Hz), 4.92 (1H, d, J=4 Hz), 4.56 (1H, d, J=4 Hz). ¹³C NMR (d₆-acetone; 400 MHz) δ 197.9, 167.6, 164.8, 164.0, 146.1 (x2), 134.0, 128.9, 107.9 (x2), 101.4, 96.8, 95.7, 84.4, 72.9. ESI-MS (positive): [M]=319.9; [M+H]=321.0; [M+H—CO—H₂O]=275.1; [M+H-2CO—H20]=247.2; [M+K]=358.9; [M+Na+MeOH]=376.8. ¹H NMR (CDCl₃, 400 MHz): δ 6.64 (s, 2H), 6.01 (s, 1H), 5.96 (s, 1H), 4.96 (d, 1H, J=3 Hz), 4.57 (d, 1H, J=3 hz). ¹³C NMR (CDCl₃, 400 MHz): δ 206.4, 197.9, 167.6, 164.8, 164.0, 146.1, 134.0, 128.9, 107.9 (2C), 101.4, 96.8, 95.7, 84.4, 72.9.

### EQUIVALENTS

Those skilled in the art will recognize, or be able to ascertain using no more than routine experimentation, many equivalents to the specific embodiments of the disclosure described herein. Such equivalents are intended to be encompassed by the following claims.

### INCORPORATION BY REFERENCE

All publications, patents, and patent applications cited herein are hereby incorporated by reference in their entirety.

1. A pure and isolated compound represented by formula I: 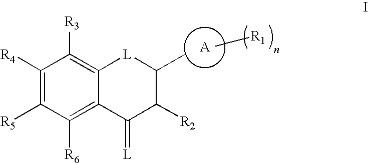
wherein, independently for each occurrence:
R₁ represents alkoxy, alkenyloxy, alkynyloxy, aryloxy, arylalkyloxy, hydroxy, —OC(O)—R₇, alkyl, alkenyl, alkynyl, acetyl, formyl, halide, cyano, nitro, SH, amino, amido, sulfonyl, or sulfonamido;
R₂ represents —OH or 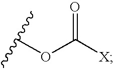
R₃, R₄, R₅, and R₆ represent H, hydroxy, alkoxy, alkenyloxy, alkynyloxy, aryloxy, aralkyloxy; —OC(O)—R₇, alkyl, alkenyl, alkynyl, aralkyl, acetyl, formyl, halide, cyano, nitro, SH, amino, amido, sulfonyl, or sulfonamido;
R₇ represents H, alkyl, alkenyl, alkynyl, cycloalkyl, aryl, arylalkyl or a carbohydrate;
A represents an aryl group;
L represents O, S, or NR;
R represents H, hydroxy, alkyl, alkenyl, alkynyl, aralkyl, acetyl, formyl, or sulfonyl;
X represents a carbohydrate, a cycloalkyl, or a cycloalkenyl group; and
n represents an integer from 1 to 5, inclusive;
wherein any of the aforementioned alkoxy, alkenyloxy, alkynyloxy, aryloxy, aralkyloxy, alkyl, alkenyl, alkynyl, cycloalkyl, cycloalkenyl, aryl and aralkyl groups may be optionally substituted with one or more groups selected from the group consisting of hydroxy, alkoxy, alkenyloxy, alkynyloxy, aryloxy, aralkyloxy; halide, formyl, acetyl, cyano, nitro, SH, amino, amido, sulfonyl, or sulfonamido.

2. The compound of claim 1, wherein, independently for each occurrence:
R₁ represents H, alkoxy, aryloxy, aralkyloxy, hydroxy, —OC(O)—R₇, alkyl, acetyl, formyl, or halide;
R₂ represents 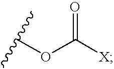
R₃, R₄, R₅, and R₆ represent H, alkoxy, aryloxy, aralkyloxy; —OC(O)—R₇, alkyl, aralkyl, acetyl, formyl, or halide;
R₇ represents H, alkyl, aryl, or arylalkyl;
A represents an aryl group;
L represents O;
X represents a carbohydrate, a cycloalkyl, or a cycloalkenyl group; and
n represents an integer from 1 to 5, inclusive;
wherein any of the aforementioned alkoxy, alkenyloxy, alkynyloxy, aryloxy, aralkyloxy, alkyl, alkenyl, alkynyl, aryl, aralkyl, cycloalkyl and cycloalkenyl groups may be optionally substituted with one or more groups selected from the group consisting of hydroxy, alkoxy, alkenyloxy, alkynyloxy, aryloxy, aralkyloxy; halide, formyl, acetyl, cyano, nitro, SH, amino, amido, sulfonyl, or sulfonamido.

3. The compound of claim 1, wherein the carbohydrate is selected from the group consisting of a monosaccharide, a disaccharide, an oligosaccharide, and a polysaccharide.

4. The compounds of claim 1, wherein R₂ is —OH.

5. The compound of claim 1, wherein L is O.

6. The compound of claim 1, wherein R₃, R₄, R₅ and R₆ are each independently H or hydroxy, wherein at least two of R₃, R₄, R₅ and R₆ are hydroxy.

7. The compound of claim 1, wherein R₁ is hydroxy, and n is equal to 2 or 3.

8. The compound of claim 1, wherein A is a benzene ring.

9. The compound of claim 1, wherein X is a carbohydrate.

10. The compound of claim 1, wherein X is a cycloalkyl or cycloalkenyl group; and wherein the cycloalkyl or cycloalkenyl group is substituted with 1 to 3 hydroxy groups.

11. A pure and isolated compound represented by formula Ia: 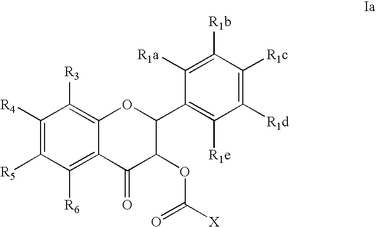
wherein, independently for each occurrence:
R₁ₐ, R_{1b}, R_{1c}, R_{1d}, R₁ₑ represent H, hydroxy, alkoxy, aralkyloxy, or aryloxy;
R₃, R₄, R₅, and R₆ represent H, hydroxy, alkoxy, aryloxy, or aralkyloxy; and
X represents a carbohydrate, a cycloalkyl, or a cycloalkenyl group;
wherein any of the aforementioned alkoxy, aryloxy, aralkyloxy may be optionally substituted with one or more groups selected from the group consisting of hydroxy, alkoxy, aryloxy, aralkyloxy; halide, formyl, acetyl, cyano, nitro, SH, amino, amido, sulfonyl, or sulfonamido.

12. The compound of claim 11, wherein: independently for each occurrence:
R₁ₐ, R_{1b}, R_{1c}, R_{1d}, and R₁ₑ represent H, hydroxy, alkoxy, aralkyloxy, or aryloxy; provided that at least two of R₁ₐ, R_{1b}, R_{1c}, R_{1d}, and R₁ₑ are hydroxy;
R₃, R₄, R₅, and R₆ represent H, hydroxy, alkoxy, aryloxy, or aralkyloxy, provided that at least two of R₃, R₄, R₅, and R₆ are hydroxy; and
X is carbohydrate, cycloalkyl, or cycloalkenyl;
wherein any of the aforementioned alkoxy, aryloxy, aralkyloxy, cycloalkyl, or cycloalkenyl may be optionally substituted with one or more groups selected from the group consisting of hydroxy, alkoxy, aryloxy, aralkyloxy; halide, formyl, acetyl, cyano, nitro, SH, amino, amido, sulfonyl, or sulfonamido.

13. The compound of claim 11, wherein:
R₁ₐ, R_{1b}, R_{1c}, R_{1d}, and R₁ₑ represent H or hydroxy, and three of R₁ₐ, R_{1b}, R_{1c}, R_{1d}, and R₁ₑ are hydroxy.

14. The compound of claim 11, wherein:
R₃, R₄, R₅, and R₆ represent H or hydroxy, and two of R₃, R₄, R₅, and R₆ are hydroxy.

15. The compound of claim 11, wherein:
X is a carbohydrate selected from the group consisting of a monosaccharide, a disaccharide, an oligosaccharide, and a polysaccharide.

16. The compound of claim 11, wherein:
X is a carbohydrate selected from the group consisting of arabinose, lyxose, ribose, rhamnose, deoxyribose, xylose, ribulose, xylulose, allose, altrose, galactose, glucose, gulose, idose, mannose, talose, fructose, psicose, sorbose, tagatose, sucrose, lactose, maltose, trehalose or cellobiose, raffinose, maltodextrin, cyclodextrin, starch, glycogen, dextran, and cellulose.

17. The compound of claim 11, wherein X is rhamnose.

18. The compound of claim 11, wherein X is a cycloalkyl or cyloalkynyl group, wherein the cycloalkyl or cycloalkenyl group is substituted with one to three hydroxy groups.

19. A pure and isolated compound represented by formula Ib: 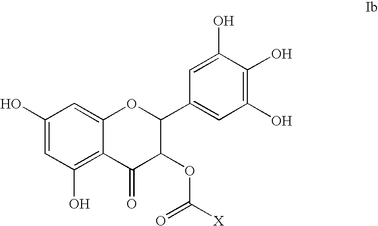
wherein X represents a carbohydrate, a cycloalkyl, or a cycloalkenyl, wherein the cycloalkyl or cycloalkenyl may be substituted with one to three hydroxy groups.

20. The compound of claim 19, wherein X is a carbohydrate selected from the group consisting of arabinose, lyxose, ribose, rhamnose, deoxyribose, xylose, ribulose, xylulose, allose, altrose, galactose, glucose, gulose, idose, mannose, talose, fructose, psicose, sorbose, tagatose, sucrose, lactose, maltose, trehalose, cellobiose, raffinose, maltodextrin, cyclodextrin, starch, glycogen, dextran, and cellulose.

21. The compound of claim 19, wherein X is a cyclohexyl or cyclohexenyl substituted with 1 to 3 hydroxy groups.

22. The compound of claim 21, wherein X is:

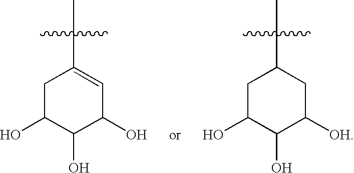

23. A pure and isolated compound selected from the group consisting of:

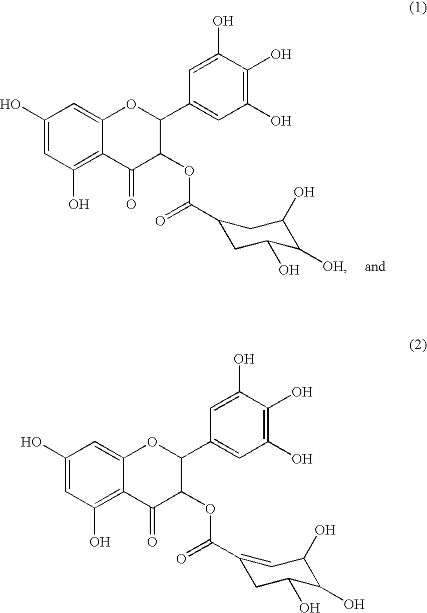

24. A pure and isolated compound of formula Ic 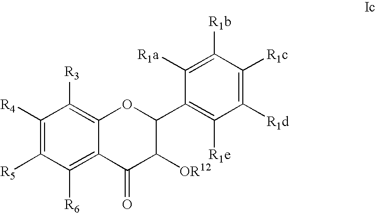
wherein, independently for each occurrence:
R₁ₐ, R_{1b}, R_{1c}, R_{1d}, R₁ₑ represent H, hydroxy, alkoxy, aralkyloxy, or aryloxy;
R₃, R₄, R₅, and R₆ represent H, hydroxy, alkoxy, aryloxy, or aralkyloxy; and
R₁₂ represents H, hydroxy, alkoxy, aralkyloxy, or aryloxy;
wherein any of the aforementioned alkoxy, aryloxy, aralkyloxy may be optionally substituted with one or more groups selected from the group consisting of hydroxy, alkoxy, aryloxy, aralkyloxy; halide, formyl, acetyl, cyano, nitro, SH, amino, amido, sulfonyl, or sulfonamido.

25. The compound of claim 24, wherein R₁ₐ, R_{1b}, R_{1c}, R_{1d}, and R₁ₑ represent H, hydroxy, alkoxy, aralkyloxy, or aryloxy; provided that at least two of R₁ₐ, R_{1b}, R_{1c}, R_{1d}, and R₁ₑ are hydroxy;
R₃, R₄, R₅, and R₆ represent H, hydroxy, alkoxy, aryloxy, or aralkyloxy, provided that at least two of R₃, R₄, R₅, and R₆ are hydroxy; and
wherein any of the aforementioned alkoxy, aryloxy, aralkyloxy, cycloalkyl, or cycloalkenyl may be optionally substituted with one or more groups selected from the group consisting of hydroxy, alkoxy, aryloxy, aralkyloxy; halide, formyl, acetyl, cyano, nitro, SH, amino, amido, sulfonyl, or sulfonamido.

26. The compound of claim 25, wherein:
R₁ₐ, R_{1b}, R_{1c}, R_{1d}, and R₁ₑ represent H or hydroxy, and three of R₁ₐ, R_{1b}, R_{1c}, R_{1d}, and R₁ₑ are hydroxy.

27. The compound of claim 25, wherein:
R₃, R₄, R₅, and R₆ represent H or hydroxy, and two of R₃, R₄, R₅, and R₆ are hydroxy.

28. The compounds of claim 24, wherein R₁₂ is OH.

29. A pure and isolated compound having the following formula:

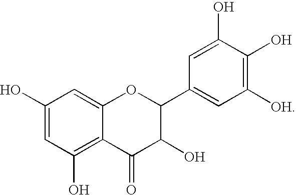

30. A pharmaceutical composition comprising a compound of claim 1 and a pharmaceutically acceptable carrier.

31. A method of treating a subject for an infection comprising administering to the subject in need thereof and effective amount of a compound of claim 1.

32. The method of claim 31, wherein the infection is a viral, bacterial, fungal, or prion infection.

33. The method of claim 32, wherein the infection is a viral infection caused by an envelope virus.

34. The method of claim 32, wherein the infection is viral infection caused by a non-envelope virus.

35. The method of claim 31, wherein the infection is a viral infection caused by an envelope virus selected from the group consisting of human influenza, avian influenza, or other influenza virus

36. The method of claim 31, wherein the infection is a bacterial infection selected from the group consisting of: Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Bordetella, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma.

37. The method of claim 31, wherein the infection is a fungal infection caused by B. cinerea, Penicillium sp., P. expansum, P. italicum, P. digitalum, Rhizopus sp., R. sulonifer, R. nigricans, Alternaria sp., A. alternata, A. solani, Diploidia sp., Diploidia natalenses, Monilinia sp., M. fructicola, Pseudomonas sp., P. cepacia, Xanthomonas sp., Erwinia sp. and Corynebacterium. Cladosporium sp., C. fulva, Phytophtora sp., P. infestans, Colletotricum spp., C. coccoides C. fragariae, C. gloesporioides, Fusarium spp., F. lycopersici, Verticillium spp., V. alboatrum, V. dahliae, Unicula spp., U. necator, Plasmopara spp., P. viticola, Guignardia spp., G. bidwellii, Cercospora spp., C. arachidicola, Scelrotinia spp., S. scerotiorum, Puccinia spp., P. arachidis, Aspergillus spp., A. favus, Venturia spp, V. inaequalis, Podosphaera spp., P. leucotricha, Pythiun spp., Sphaerotheca, or S. macularis.

38. The method of claim 31, wherein the infection is prion infection selected from the group consisting of scrapie in sheep, bovine spongiform encephalopathy (BSE), transmissible mink encephalopathy (TME), chronic wasting disease (CWD) in elk and mule deer, feline spongiform encephalopathy in cats, exotic ungulate encephalopathy (EUE) in nyala, oryx, and greater kudu, Creutzfeldt-Jakob Disease (CJD), Iatrogenic Creutzfeldt-Jakob disease, Variant Creutzfeldt-Jakob disease, Familial Creutzfeldt-Jakob disease, Sporadic Creutzfeldt-Jakob disease; Gerstmann-Straussler-Scheinker syndrome (GSS), Fatal Familial Insomnia (FFI), Kuru, and Alpers syndrome.

39. The method of claim 31 wherein the infection is protozoan infection selected from the group consisting of Entamoeba histolytica, Giardia lambila, Trichomonas vaginalis, Trypanosoma brucei T. cruzi, Leishmania donovani, Balantidium coli, Toxoplasma gondii, Plasmodium spp., Babesia microti, sleeping sickness (Trypanosomeniasis), Amoebiasis, Giardiasis, Trichomoniasis, African Sleeping Sickness, American Sleeping Sickness, Leishmaniasis, Balantidiasis, Toxoplasmosis, Malaria, and Babesiosis.

40. The method of claim 31, wherein the subject is a mammal.

41. The method of claim 31, wherein the subject is a primate.

42. The method of claim 31, wherein the subject is human.

43. The method of claim 31, wherein the subject is a fowl family Avves.

44. The method of claim 31, wherein the subject is a swine family porcine.

45. A method of preparing a compound of claim 1, comprising extracting a botanical with water to obtain an eluate, loading the eluate onto a filtering agent, washing the eluate with a buffer to provide a filtering agent-bound fraction, and releasing the filtering agent bound fraction with a high ionic strength buffer.

46. A method of making a compound of claim 1, comprising:
a) reacting an acetylphenone with a benzaldehyde to form a chalcone;
b) epoxidizing the chalcone to form an epoxide; and
c) cyclizing the epoxide to form a flavononol.

47. A method of making a compound of claim 1, comprising:
a) reacting an acetylphenone with a benzaldehyde to form a chalcone;
b) cyclizing the chalcone to form a flavanone; and
c) oxidizing the flavanone to yield a flavononol.

48. The method of claim 46, further comprising esterifying the dihydroflavonol to yield an esterified flavononol.

49. A method of making a vaccine base on the binding site of a compound of claim 1 comprising
a. the binding site amino acid sequence that numbers 3-7 amino acids
b. the binding site amino acid sequence the encompasses the 10 Å binding site of the compounds
c. utilizing the binding site sequence as an antigen for antibody and vaccine production.

50. A diagnostic comprising a compound of claim 1 tethered to a plate.

51. The diagnostic of claim 45, wherein the compound is tethered via an ester or amide linkage to the A ring of the compound.

52. A diagnostic comprising a compound of claim 1 in a solution.

53. A method of identifying a pathogen or amyloid, comprising:
a) incubation of a sample suspected of containing the pathogen or amyloid with a compound of claim 1 in a solvent to form a mixture;
b) filtering the mixture with a membrane filter to remove unbound compounds;
c) detecting the compound using DART TOF-MS analysis.

54. A biodefense filter comprising a compound of claim 1.

55. The biodefense filter of claim 54, wherein the filter is incorporated into a facial mask.

56. The biodefense filter of claim 54, wherein the filter is incorporated into an article of clothing.

57. The biodefense filter of claim 54, wherein the filter is incorporated into an HVAC system.

58. The biodefense filter of claim 54, wherein the filter is incorporated into a water treatment system.

59. The method of claim 36, wherein the bacterial infection is selected from the group consisting of Streptococcus pneumoniae, Streptococcus pyogenes, Streptococcus agalactiae, Streptococcus faecalis, Streptococcus faecium, Streptococcus durans, Neisseria gonorrhea, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Burkholderia cepacia, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii, Helicobacter pylori and Chlamydia trachomitis.
